(19) United States
(12) Patent Application Publication
(10) Pub. No.: US 2025/0259034 A1
(43) Pub. Date: Aug. 14, 2025

(54) Deep Learning for Non-compartmental Analysis

(71) Applicant: Genentech, Inc., South San Francisco, CA (US)

(72) Inventors: Jin Yan Jin, Foster City, CA (US); Gengbo Liu, Newark, CA (US); James Lu, San Francisco, CA (US)

(21) Appl. No.: 19/191,289

(22) Filed: Apr. 28, 2025

Related U.S. Application Data
(63) Continuation of application No. PCT/US2023/077559, filed on Oct. 23, 2023.
(60) Provisional application No. 63/420,452, filed on Oct. 28, 2022.

(51) Int. Cl. G06N 3/045; G16B 5/00; G16B 40/20

(52) U.S. Cl. CPC G06N 3/045; G16B 5/00; G16B 40/20

(57) ABSTRACT

A method may include training a machine learning model to determine a first pharmacokinetic parameter and a second pharmacokinetic parameter for a molecule. The machine learning model may be trained by at least determining, based at least on an input including one or more pharmacokinetic models associated with the molecule, a first value of the first pharmacokinetic parameter, determining, based at least on the input, a second value of the first pharmacokinetic parameter, and determining, based at least on the first value and the second value of the first pharmacokinetic parameter, a third value of the second pharmacokinetic parameter. The method may also include applying the trained machine learning model to determine, based at least on a sparsely sampled pharmacokinetic model associated with the molecule, the first pharmacokinetic parameter and/or the second pharmacokinetic parameter. Related methods and articles of manufacture are also disclosed.

### PRIORITY

This application is a continuation under 35 U.S.C. § 365 (c) of International Patent Application No. PCT/US2023/077559, filed 23 Oct. 2023, which claims the benefit under 35 U.S.C. § 119 (e) of U.S. Provisional Patent Application No. 63/420,452, filed 28 Oct. 2022, each of which is incorporated herein by reference.

### TECHNICAL FIELD

The present disclosure generally relates to machine learning and more specifically to deep learning for non-compartmental analysis.

### BACKGROUND

Pharmacokinetics can be used to describe an absorption, distribution, metabolism, and excretion of a molecule. Pharmacokinetic parameters are used to model the activity of the molecule within the body. For example, pharmacokinetic parameters, such as an area under curve, a maximum plasma concentration of the molecule, and a half-life of the molecule, play an important role in designing, refining, and creating safe and effective therapeutics. Generally, non-compartmental analysis has been used to calculate such pharmacokinetic parameters. Current approaches for estimating pharmacokinetic parameters, especially in cases involving sparsely sampled measurements, are often rudimentary and result in inaccurate determinations. The following description provides solution(s) to these problems and provide additional benefits as well.

### SUMMARY OF PARTICULAR EMBODIMENTS

Methods, systems, and articles of manufacture, including computer program products, are provided for deep learning for non-compartmental analysis. In one aspect, there is provided a system. The system may include at least one processor and at least one memory. The at least one memory may store instructions that result in operations when executed by the at least one processor. The operations may include: training a machine learning model to determine a first pharmacokinetic parameter and a second pharmacokinetic parameter for a molecule by at least: determining, based at least on an input including one or more pharmacokinetic models associated with the molecule, a first value of the first pharmacokinetic parameter, determining, based at least on the input, a second value of the first pharmacokinetic parameter, and determining, based at least on the first value and the second value of the first pharmacokinetic parameter, a third value of the second pharmacokinetic parameter. The operations may also include applying the trained machine learning model to determine, based at least on a sparsely sampled pharmacokinetic model associated with the molecule, the first pharmacokinetic parameter and/or the second pharmacokinetic parameter.

In some variations, one or more of the features disclosed herein including the following features can optionally be included in any feasible combination. In some variations, the input further includes one or more ground truth target values for the first pharmacokinetic parameter and/or the second pharmacokinetic parameter associated with the one or more pharmacokinetic models, The one or more ground truth values are determined by at least applying non-compartmental analysis to the one or more pharmacokinetic models. The machine learning model is further trained by at least minimizing an error between the third value of the second pharmacokinetic parameter and a ground truth target value of the one or more ground truth target values corresponding to the second pharmacokinetic parameter.

In some variations, the one or more pharmacokinetic models includes time series data of a plasma concentration of the molecule collected at a plurality of time points after the molecule is delivered to a patient.

In some variations, the time series concentration data includes: a first tuple including a first time point of the plurality of time points, a plasma concentration measured at the first time point, and a dosage measured at the first time point, and a second tuple including a second time point of the plurality of time points, a second plasma concentration measured at the second time point, and a second dosage measured at the second time point.

In some variations, the first value is determined at a first time point and the second value is determined at a second time point after the first time point.

In some variations, the first pharmacokinetic parameter is an area under curve (AUC) of a plasma concentration curve associated with the one or more pharmacokinetic models.

In some variations, the first value corresponds to a first area under curve at the first time point and the second value corresponds to a second area under curve at the second time point.

In some variations, the second pharmacokinetic parameter is at least one of a maximum plasma concentration of the molecule and a half-life of the molecule.

In some variations, the first value and the second value are determined by at least one convolutional layer of the machine learning model.

In some variations, the at least one convolutional layer includes two convolutional layers.

In some variations, the third value is determined by at least one recurrent neural network unit of the machine learning model.

In some variations, the at least one recurrent neural network unit includes two recurrent neural network units.

In some variations, the machine learning model is further trained to determine the one or more pharmacokinetic parameters for the molecule by at least: outputting, by the at least one convolutional layer, the first value and the second value and receiving, by the at least one recurrent neural network unit, the first value and the second value.

In some variations, the machine learning model is further trained to determine the one or more pharmacokinetic parameters for the molecule by at least determining, based on the first value and the second value, a fourth value of a third pharmacokinetic parameter of the one or more pharmacokinetic parameters.

In some variations, the molecule is a micro molecule having a molecular weight of less than 1000 Daltons.

In some variations, the molecule is a macro molecule having a molecular weight of greater than or equal to 1000 Daltons.

In some variations, the sparsely sampled pharmacokinetic model associated with the molecule includes a concentration curve across a plurality of doses of the molecule.

In some variations, the operations include normalizing a unit of measurement associated with the one or more pharmacokinetic models.

In some variations, the one or more pharmacokinetic models includes a densely sampled pharmacokinetic model in which a plasma concentration of the molecule is collected at an above-threshold frequency.

In some variations, the one or more pharmacokinetic models includes a sparsely sampled pharmacokinetic model in which a plasma concentration of the molecule is collected at a below-threshold frequency.

In another aspect, there is provided a method for non-compartmental analysis. The method may include: training a machine learning model to determine a first pharmacokinetic parameter and a second pharmacokinetic parameter for a molecule by at least: determining, based at least on an input including one or more pharmacokinetic models associated with the molecule, a first value of the first pharmacokinetic parameter, determining, based at least on the input, a second value of the first pharmacokinetic parameter, and determining, based at least on the first value and the second value of the first pharmacokinetic parameter, a third value of the second pharmacokinetic parameter. The method may also include applying the trained machine learning model to determine, based at least on a sparsely sampled pharmacokinetic model associated with the molecule, the first pharmacokinetic parameter and/or the second pharmacokinetic parameter.

In another aspect, there is provided a computer program product that includes a non-transitory computer readable storage medium. The non-transitory computer-readable storage medium may include program code that causes operations when executed by at least one processor. The operations may include: training a machine learning model to determine a first pharmacokinetic parameter and a second pharmacokinetic parameter for a molecule by at least: determining, based at least on an input including one or more pharmacokinetic models associated with the molecule, a first value of the first pharmacokinetic parameter, determining, based at least on the input, a second value of the first pharmacokinetic parameter, and determining, based at least on the first value and the second value of the first pharmacokinetic parameter, a third value of the second pharmacokinetic parameter. The operations may also include applying the trained machine learning model to determine, based at least on a sparsely sampled pharmacokinetic model associated with the molecule, the first pharmacokinetic parameter and/or the second pharmacokinetic parameter.

In another aspect, there is provided a system. The system may include at least one processor and at least one memory. The at least one memory may store instructions that result in operations when executed by the at least one processor. The operations may include: normalizing a first value of a first pharmacokinetic measurement associated with a first pharmacokinetic model by at least: applying a first scaling factor to the first value to generate a first normalized value. The first value is in a first unit of measurement, and the first normalized value is unitless. The operations may also include normalizing a second value of a second pharmacokinetic measurement associated with the first pharmacokinetic model by at least: applying a second scaling factor to the second value to generate a second normalized value. The second value is in a second unit of measurement, and the second normalized value is unitless. The operations may further include training a machine learning model to determine one or more pharmacokinetic parameters for a molecule associated with one or more pharmacokinetic models including the first pharmacokinetic model based at least on an input including the first normalized value and the second normalized value.

In some variations, the first scaling factor is a first maximum value of a plurality of first values of the first pharmacokinetic measurement across the first pharmacokinetic model and the second scaling factor is a second maximum value of a plurality of second values of the second pharmacokinetic measurement across the first pharmacokinetic model.

In some variations, the operations include normalizing a third value of a third pharmacokinetic measurement associated with the first pharmacokinetic model by at least: applying a third scaling factor to the third value to generate a third normalized value. The third value is in a third unit of measurement, and the third normalized value is unitless.

In some variations, the operations include normalizing a first target value of a first target pharmacokinetic parameter associated with the first pharmacokinetic model by at least: applying the first scaling factor to the first target value of the first target pharmacokinetic parameter. The first target value is in a first target unit of measurement, the first unit of measurement is the same as the first target unit of measurement, and the normalized first target value is unitless.

In some variations, the first target pharmacokinetic parameter is one of a maximum plasma concentration of the molecule and a half-life of the molecule.

In some variations, the operations include normalizing a second target value of a second target pharmacokinetic parameter associated with the first pharmacokinetic model by at least: applying the second scaling factor to the second target value of the second target pharmacokinetic parameter. The second target value is in a second target unit of measurement, the second unit of measurement is the same as the second target unit of measurement, and the normalized second target value is unitless.

In some variations, the operations include: normalizing a third target value of a third target pharmacokinetic parameter associated with the first pharmacokinetic model by at least: applying a third scaling factor to the third target value of the third target pharmacokinetic parameter. The third scaling factor is generated by at least multiplying the first scaling factor and the second scaling factor.

In some variations, the third target pharmacokinetic parameter is an area under curve (AUC) of a plasma concentration curve associated with the first pharmacokinetic model.

In some variations, the first pharmacokinetic measurement is a first time point and the second pharmacokinetic measurement is a plasma concentration measured at the first time point.

In some variations, the input further includes the first target value.

In some variations, the applying the first scaling factor to the first value includes dividing the first value by the first scaling factor. The applying the second scaling factor to the second value includes dividing the second value by the second scaling factor.

In another aspect, there is provided a method for non-compartmental analysis. The method may include: normalizing a first value of a first pharmacokinetic measurement associated with a first pharmacokinetic model by at least: applying a first scaling factor to the first value to generate a first normalized value. The first value is in a first unit of measurement, and the first normalized value is unitless. The method may also include normalizing a second value of a second pharmacokinetic measurement associated with the first pharmacokinetic model by at least: applying a second scaling factor to the second value to generate a second normalized value. The second value is in a second unit of measurement, and the second normalized value is unitless. The method may further include training a machine learning model to determine one or more pharmacokinetic parameters for a molecule associated with one or more pharmacokinetic models including the first pharmacokinetic model based at least on an input including the first normalized value and the second normalized value.

In another aspect, there is provided a computer program product that includes a non-transitory computer readable storage medium. The non-transitory computer-readable storage medium may include program code that causes operations when executed by at least one processor. The operations may include: normalizing a first value of a first pharmacokinetic measurement associated with a first pharmacokinetic model by at least: applying a first scaling factor to the first value to generate a first normalized value. The first value is in a first unit of measurement, and the first normalized value is unitless. The operations may also include normalizing a second value of a second pharmacokinetic measurement associated with the first pharmacokinetic model by at least: applying a second scaling factor to the second value to generate a second normalized value. The second value is in a second unit of measurement, and the second normalized value is unitless. The operations may further include training a machine learning model to determine one or more pharmacokinetic parameters for a molecule associated with one or more pharmacokinetic models including the first pharmacokinetic model based at least on an input including the first normalized value and the second normalized value.

In another aspect, there is provided a system. The system may include at least one processor and at least one memory. The at least one memory may store instructions that result in operations when executed by the at least one processor. The operations may include: simulating a plurality of pharmacokinetic profiles for each dosing scenario of a plurality of dosing scenarios using a first model; generating dense pharmacokinetic profiles with a first sampling rate; calculating ground truth non-compartmental analysis (NCA) data for pharmacokinetic parameters from the dense pharmacokinetic profiles using a non-compartmental analysis tool; subsampling from the dense pharmacokinetic profiles to generate typical pharmacokinetic data; and training the deep learning model using pairs of the typical pharmacokinetic data and ground truth NCA data.

In some variations, the dosing scenarios comprise a 21-day dosing interval for large molecules and a daily dosing for small molecules.

In some variations, the dosing scenarios comprise intravenous, subcutaneous, and oral administration of a molecule.

In some variations, the ground truth NCA data includes the calculation of half-life, area under the curve after the last dose (AUClast), and peak plasma concentration (Cmax) of a molecule.

In some variations, the subsampling from the dense pharmacokinetic profiles includes establishment of discretized time bins based on the Time After Dose (TAD) and the random selection of one pharmacokinetic sample per bin for each dose.

In some variations, the deep learning model is a Convolutional Recurrent Neural Network (CRNN) that is trained using an Adam optimizer.

In some variations, the operations further comprise evaluating a predictive performance of the deep learning model by comparing the predicted non-compartmental pharmacokinetic parameters with actual pharmacokinetic parameters.

In some variations, the evaluation of the predictive performance of the deep learning model includes the use of R2(coefficient of determination), normalized root mean square deviation (nRMSE), and mean absolute percentage error (MAPE) as measures of the predictive performance.

In some variations, the deep learning model is separately trained on pharmacokinetic parameter distributions tailored to small and large molecules.

In some variations, the deep learning model is trained on both the 36original dense typical pharmacokinetic data and sparsified versions of the typical pharmacokinetic data.

In another aspect, there is provided a system. The system may comprise: a simulation module configured to simulate a plurality of pharmacokinetic profiles for each dosing scenario of a plurality of dosing scenarios using a first model; a generation module configured to generate dense pharmacokinetic profiles with a first sampling rate; a calculation module configured to calculate ground truth non-compartmental analysis (NCA) data for pharmacokinetic parameters from the dense pharmacokinetic profiles using a non-compartmental analysis tool; a subsampling module configured to subsample from the dense pharmacokinetic profiles to generate typical pharmacokinetic data; and a training module configured to train the deep learning model using pairs of the typical pharmacokinetic data and ground truth NCA data.

In some variations, the dosing scenarios comprise a 21-day dosing interval for large molecules and a daily dosing for small molecules.

In some variations, the dosing scenarios comprise intravenous, subcutaneous, and oral administration of a molecule.

In some variations, the calculation module is configured to calculate ground truth NCA data including the half-life, area under the curve after the last dose (AUClast), and peak plasma concentration (Cmax) of a molecule

In some variations, the subsampling module is configured to establish discretized time bins based on the Time After Dose (TAD) and to randomly select one pharmacokinetic sample per bin for each dose.

In some variations, the deep learning model is a Convolutional Recurrent Neural Network (CRNN) that is trained using an Adam optimizer.

In some variations, the system further comprises an evaluation module configured to evaluate the predictive performance of the deep learning model by comparing the predicted non-compartmental pharmacokinetic parameters with actual pharmacokinetic parameters.

In some variations, the evaluation module is configured to use R2 (coefficient of determination), normalized root mean square deviation (nRMSE), and mean absolute percentage error (MAPE) as measures of the predictive performance.

In some variations, the deep learning model is separately trained on pharmacokinetic parameter distributions tailored to small and large molecules.

In some variations, the deep learning model is trained on both the original dense typical pharmacokinetic data and sparsified versions of the typical pharmacokinetic data.

In another aspect, there is provided a method for evaluating the predictive performance and generalization of a deep learning model for non-compartmental analysis (Deep-NCA model), the method comprising: simulating a test dataset consisting of a plurality of anonymized drugs, each drug associated with a distinct pharmacokinetic profile and a population of patients; generating, for each drug in the test dataset, pairs of typical pharmacokinetic data and corresponding ground truth non-compartmental analysis (NCA) data using a corresponding population pharmacokinetic model; modifying the typical pharmacokinetic data to create sparse pharmacokinetic profiles by randomly omitting a predetermined number of time points from the typical pharmacokinetic data; and comparing a performance of the Deep-NCA model in predicting pharmacokinetic parameters based on the sparse pharmacokinetic profiles to a performance of a traditional non-compartmental analysis performed on the sparse pharmacokinetic profiles.

In some variations, the test dataset is simulated to comprise a variety of pharmacokinetic profiles representing a diverse range of drug types, including small molecule drugs, large molecule drugs, monoclonal antibodies, and antibody-drug conjugates.

In some variations, the predetermined number of time points omitted from the typical pharmacokinetic data is determined based on the desired level of sparsity in the pharmacokinetic profiles.

In some variations, the performance comparison includes the use of statistical measures such as R2 (coefficient of determination), normalized root mean square deviation (nRMSE), and mean absolute percentage error (MAPE) to evaluate the accuracy of the Deep-NCA model's predictions.

In some variations, the traditional non-compartmental analysis performed on the sparse pharmacokinetic profiles is conducted using a standard non-compartmental analysis tool.

In some variations, the performance of the Deep-NCA model is further evaluated by comparing its predictions with the ground truth NCA data for the test dataset.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to deep learning for non-compartmental analysis, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts an example pharmacokinetic prediction system, consistent with implementations of the current subject matter;

FIG. 2A depicts a network diagram illustrating a pharmacokinetic prediction system, consistent with implementations of the current subject matter;

FIG. 2B schematically depicts a preprocessing process, consistent with implementations of the current subject matter;

FIG. 2C schematically depicts a visual representation of the patient-specific normalization process, consistent with implementations of the current subject matter;

FIG. 3 depicts an example input for a machine learning model for predicting pharmacokinetic parameters;

FIG. 4 depicts a performance comparison, consistent with implementations of the current subject matter;

FIG. 5 depicts a flowchart illustrating an example of a process for normalizing an input for a machine learning model, consistent with implementations of the current subject matter.

FIG. 6A depicts a flowchart illustrating an example of a process for deep learning for non-compartmental analysis, consistent with implementations of the current subject matter;

FIG. 6B depicts a flowchart illustrating an example of a process for deep learning for non-compartmental analysis, consistent with implementations of the current subject matter;

FIG. 7 depicts a block diagram illustrating an example of a computing system, consistent with implementations of the current subject matter;

FIG. 8 depicts an architecture illustrating a pharmacokinetic prediction system, consistent with implementations of the current subject matter;

FIG. 9 depicts a flowchart illustrating an example of a process for training a deep learning model, consistent with implementations of the current subject matter.

When practical, like labels are used to refer to same or similar items in the drawings.

### DESCRIPTION OF EXAMPLE EMBODIMENTS

Pharmacokinetic parameters, such as an area under curve, a maximum plasma concentration of a molecule, and a half-life of the molecule, play an important role in designing, refining, and creating safe and effective therapeutics and treatment protocols for delivering such therapeutics. Generally, non-compartmental analysis has been used to calculate such pharmacokinetic parameters. Non-compartmental analysis numerically infers the pharmacokinetic parameters. For example, using non-compartmental analysis, an area under curve of a plasma concentration curve for a pharmacokinetic model can conventionally be estimated using numerical approaches, such as the Trapezoid Rule, in which the area under curve is determined as a sum of a trapezoidal area under the concentration curve. The half-life and the maximum plasma concentration of the molecule may also generally be determined by examining the concentration curve for the particular pharmacokinetic model and by comparing the collected pharmacokinetic measurements. However, such approaches are often rudimentary and do not accurately estimate the pharmacokinetic parameters in circumstances in which the pharmacokinetic models include sparsely sampled pharmacokinetic measurements and pharmacokinetic measurements that are collected at variable time points. Using non-compartmental analysis in such circumstances often leads to inaccurate pharmacokinetic parameter determinations.

Moreover, rather than using non-compartmental analysis to determine the pharmacokinetic parameters, machine learning models have been used to more quickly estimate the pharmacokinetic parameters. However, such machine learning models similar fail to accurately estimate the pharmacokinetic parameters in various circumstances, such as when the pharmacokinetic models are sparsely sampled. Pharmacokinetic models are sparsely sampled, as described in more detail herein, when the pharmacokinetic models include a limited quantity of pharmacokinetic measurements that can be used for predicting the pharmacokinetic parameters. Thus, sparsely sampled pharmacokinetic models provides incomplete data that makes it difficult for the machine learning models to accurately and efficiently predict the pharmacokinetic parameters.

Generally, machine learning models for predicting pharmacokinetic parameters may also require the input sequences to have a particular size and/or length. In other words, conventional machine learning models may require input sequences having pharmacokinetic measurements taken at a particular quantity of time points. However, such machine learning models similarly fail to accurately and effectively predict pharmacokinetic parameters when the input sequences have a variable length, such as when the input sequences include a variable quantity of pharmacokinetic measurements collected at a varying quantity of time points.

Further, the inputs to machine learning models for predicting pharmacokinetic parameters are generally not standardized. For example, the input pharmacokinetic measurements underlying the pharmacokinetic models may have different units depending on the patient, the method of collecting the pharmacokinetic measurements, the devices that collected the pharmacokinetic measurements, and/or the like. Such non-standardized measurements can make pharmacokinetic parameter predictions computationally expensive and inefficient. Such non-standardized measurements may also lead to inaccurate predictions of pharmacokinetic parameters.

Consistent with implementations of the current subject matter, a pharmacokinetic prediction system trains a machine learning model to accurately and efficiently predict pharmacokinetic parameters, such as an area under curve of a plasma concentration curve associated with the one or more pharmacokinetic models, a maximum plasma concentration of the molecule, a half-life of the molecule, and/or the like. For example, the machine learning model may include at least one convolutional layer followed by at least one recurrent neural network unit. As described herein, the at least one convolutional layer captures a snapshot of the pharmacokinetic measurements at a particular time, while the at least one recurrent neural network unit propagates the captured pharmacokinetic measurements over time across time series concentration data. The machine learning model consistent with implementations of the current subject matter may thus accurately predict pharmacokinetic parameters for both sparsely sampled pharmacokinetic models and densely sampled pharmacokinetic models. The machine learning model consistent with implementations of the current subject matter may additionally and/or alternatively accurately predict the pharmacokinetic parameters, regardless of the length of the input sequence and/or the number of dimensions of the input sequence. Additionally and/or alternatively, the pharmacokinetic prediction system may normalize the input for the machine learning model such that the input is unitless. Such configurations may improve the accuracy and/or efficiency of the machine learning model, while reducing the required computational resources.

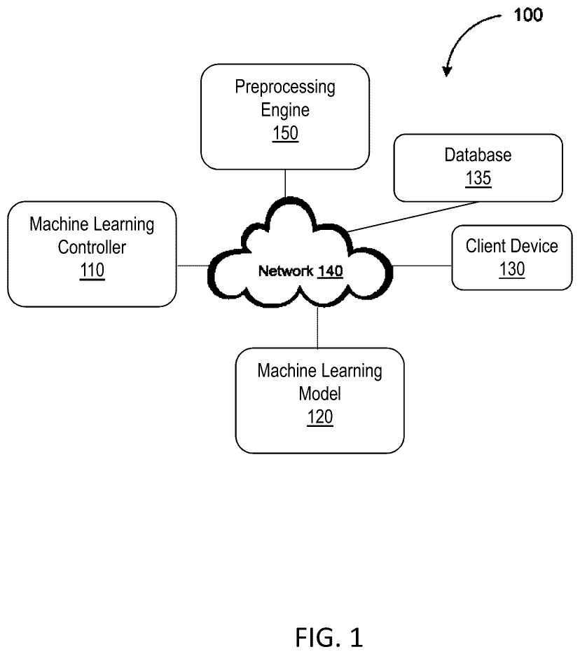

FIG. 1 depicts a system diagram illustrating an example of a pharmacokinetic prediction system 100, consistent with implementations of the current subject matter. Referring to FIG. 1, the pharmacokinetic prediction system 100 may include a machine learning controller 110, a machine learning model 120, a preprocessing engine 150, a database 135, and a client device 130. The machine learning controller 110, the machine learning model 120, the preprocessing engine 150, the database 135, and the client device 130 may be communicatively coupled via a network 140. The network 140 may be a wired network and/or a wireless network including, for example, a wide area network (WAN), a local area network (LAN), a virtual local area network (VLAN), a public land mobile network (PLMN), the Internet, and/or the like. In some implementations, the machine learning controller 110, the machine learning model 120, the preprocessing engine 150, the database 135, and/or the client device 130 may be contained within and/or operate on a same device. It should be appreciated that the client device 130 may be a processor-based device including, for example, a smartphone, a tablet computer, a wearable apparatus, a virtual assistant, an Internet-of-Things (IoT) appliance, and/or the like. The machine learning controller 110 includes at least one data processor and at least one memory storing instructions, which when executed by the at least one data processor, perform one or more operations as described herein.

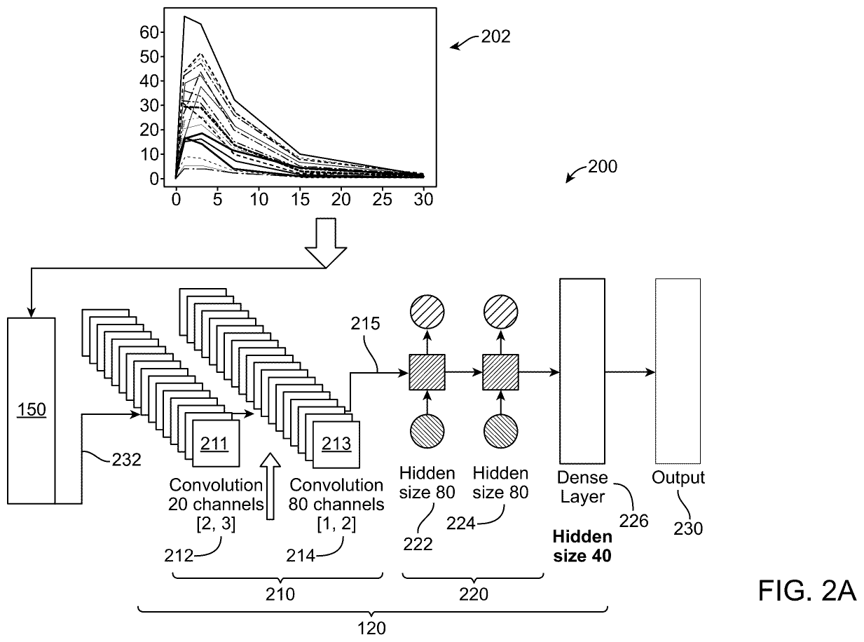

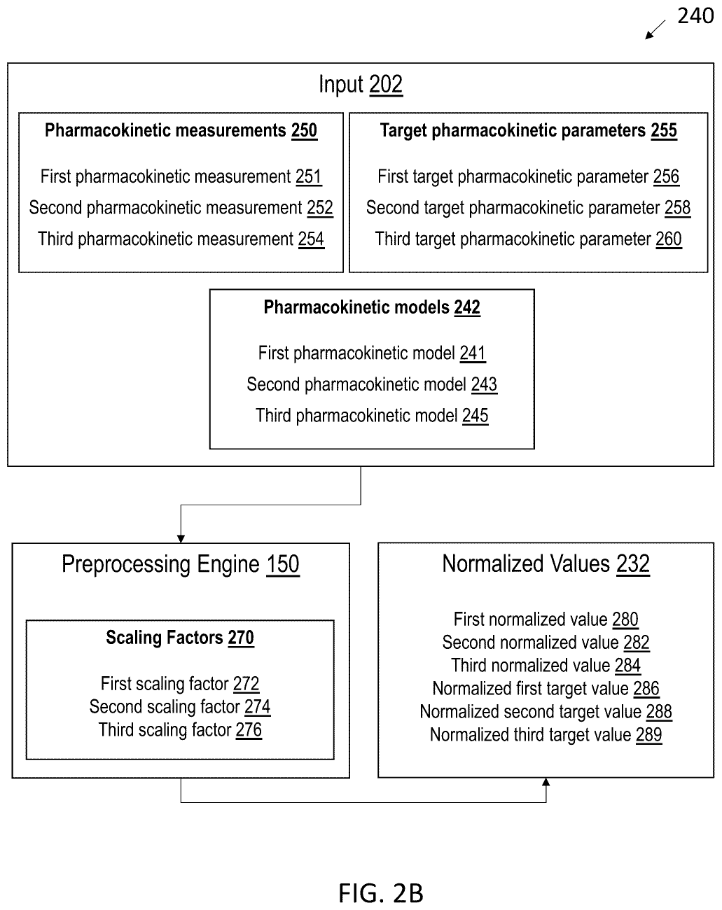

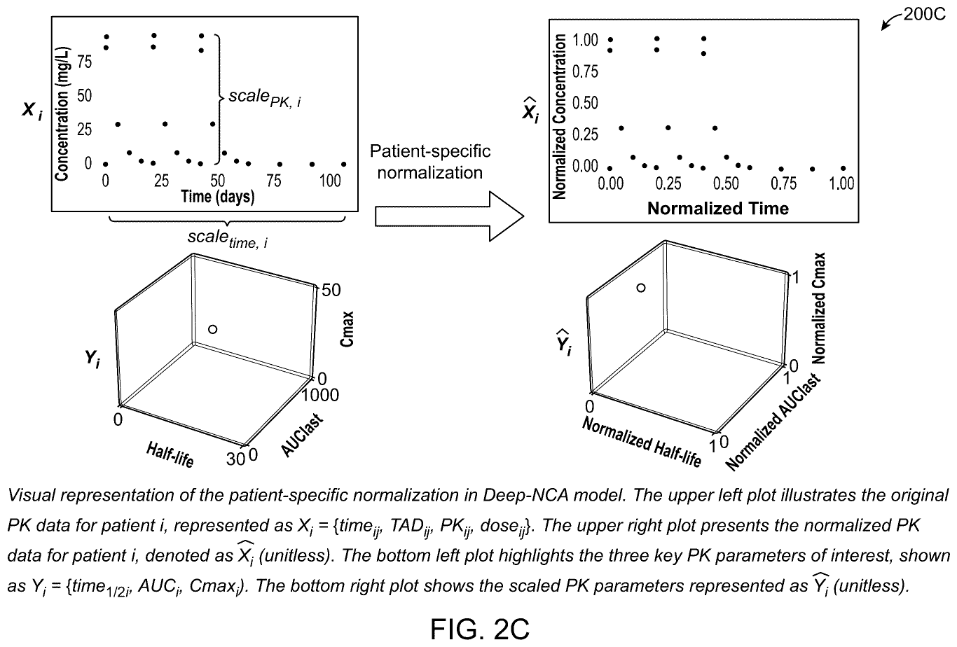

FIG. 2A schematically illustrates an example architecture 200 of the machine learning model 120. The machine learning controller 110 may train the machine learning model 120, using the architecture 200 and based on an input 202, to determine one or more pharmacokinetic parameters for a molecule. The molecule is at least one of a micro molecule having a molecular weight of less than 1000 Daltons and a macro molecule having a molecular weight of greater than or equal to 1000 Daltons. The molecule may be delivered to a patient to achieve a desired therapeutic effect. The one or more pharmacokinetic parameters may be used to determine an activity of the molecule in the body of the patient, which can in turn be used to determine the efficacy and/or therapeutic effect of the molecule. The one or more pharmacokinetic parameters may additionally and/or alternatively be used for molecule design.

The one or more pharmacokinetic parameters may include an area under curve (also referred to as “AUC”), a maximum plasma concentration (also referred to as “Cₘₐₓ”) of a molecule, a minimum plasma concentration (also referred to as “Cₘᵢₙ”), a half-life (also referred to as “t_{1/2}”) of a molecule, a time of maximum concentration (also referred to as “Tₘₐₓ”) of a molecule, a mean residence time (also referred to as “MRT”), and/or the like.

The area under curve may be an area under a plasma concentration curve associated with one or more pharmacokinetic models 242 (see FIG. 2B), such as a first pharmacokinetic model 241, a second pharmacokinetic model 243, a third pharmacokinetic model 245, and/or the like. The plasma concentration curve may describe plasma concentration in the blood plasma of the patient as a function of time after a dose of a molecule is delivered to the patient. The maximum plasma concentration of the molecule may refer to a maximum (e.g., a peak) plasma concentration in the blood plasma of the patient after delivery of the molecule to the patient. The maximum plasma concentration of the molecule may refer to the maximum plasma concentration after a single dose and/or after multiple doses of the molecule. The minimum plasma concentration of the molecule may refer to a minimum (e.g., a trough) plasma concentration in the blood plasma of the patient after delivery of the molecule to the patient. The minimum plasma concentration of the molecule may refer to the minimum plasma concentration after a single dose and/or after multiple doses of the molecule. The time of maximum concentration of the molecule may refer to a time point associated with the maximum plasma concentration. In other words, the time of maximum concentration may be a time point at which the plasma concentration in the blood plasma of the patient has reached the maximum plasma concentration after a single dose and/or multiple doses of the molecule. The half-life of the molecule may refer to a time for a dose of the molecule in the blood plasma of the patient after delivery of the molecule to the patient to reach one-half of its steady-state value. The mean residence time may refer to an average time the molecule remains in the body of the patient after delivery of a single dose and/or multiple doses of the molecule to the patient.

The machine learning controller 110 may train the machine learning model 120 using the architecture 200 and based on the input 202. The database 135 may store the input 202. The input 202 includes one or more (e.g., one, two, three, four, five, six, seven, eight, nine, ten or more) pharmacokinetic models 242 associated with a molecule (see FIG. 2B). The one or more pharmacokinetic models 242 include time series data of a plasma concentration collected at a plurality of time points after the molecule is delivered to a patient. Thus, the one or more pharmacokinetic models 242 can be used to model the activity of the molecule within the body after delivery of the molecule to the patient. The time series concentration data includes a plurality of pharmacokinetic measurements 250 taken at a plurality of time points (see FIG. 2B). The pharmacokinetic measurements 250 may include a time point, a plasma concentration measured at the time point, a dosage measured at the time point, and/or the like. The one or more pharmacokinetic models 242 and/or the underlying plurality of pharmacokinetic measurements 250 can be stored in the database 135.

The time series concentration data may form at least a portion of the input 202 as a plurality of tuples defining an inputs sequence. The input sequence may include the plurality of tuples. Each tuple of the plurality of tuples may include a value of the plurality of pharmacokinetic measurements 250. For example, the tuple may be represented as X={time point, plasma concentration, dose}. Consistent with implementations of the current subject matter, the architecture 200 is configured to receive an input sequence (e.g., the input 202) having any length. In other words, the input sequence may include any number of tuples and/or may not be limited to a particular quantity of tuples. Such configurations may desirably allow for the machine learning model 120 to determine one or more pharmacokinetic parameters based on sparsely sampled pharmacokinetic models (described in more detail below).

The one or more pharmacokinetic models 242 of the input 202 may include at least one densely sampled pharmacokinetic model and/or at least one sparsely sampled pharmacokinetic model. The at least one densely sampled pharmacokinetic model and/or the at least one sparsely sampled pharmacokinetic model may be generated based on molecule activity in the body of the patient after a single dose of the molecule has been delivered to the patient and/or after multiple doses of the molecule have been delivered to the patient.

As described herein, the at least one densely sampled pharmacokinetic model may include a plasma concentration of the molecule collected at an above-threshold frequency (e.g., a frequency that meets the threshold frequency) and/or an above-threshold quantity (e.g., a quantity that meets the threshold quantity) of pharmacokinetic measurements that is sufficient to describe the activity of the molecule after delivery to the patient. The above-threshold frequency may include pharmacokinetic measurements collected at every 1 hour, 1 minute to 30 minutes, 30 minutes to 1 hour, 1 hour to 1.5 hours, 1.5 hours to 2.0 hours, 12 hours to 24 hours, 24 hours to 48 hours, 24 hours to 1 week, and/or the like. The above-threshold quantity may include pharmacokinetic measurements collected at 10-12 time points, at least 10 time points, at least 3 time points, at least 5 to 10 time points, and/or the like. Additionally and/or alternatively, the at least one densely sampled pharmacokinetic model may include a plurality of distinct pharmacokinetic measurements, such as maximums (e.g., peaks) and minimums (e.g., troughs), following at least one dose of the molecule delivered to the patient. The plurality of distinct pharmacokinetic measurements may be unique relative to one another. For example, the distinct pharmacokinetic measurements (e.g., the peaks and/or troughs) may have values that are sufficiently different from one another such that the values can be considered as part of the pharmacokinetic measurements collected at the above-threshold frequency and/or the above-threshold quantity. This helps to ensure that the collected pharmacokinetic measurements are sufficient to describe the activity of the molecule after delivery to the patient. As described herein, the machine learning controller 110 may use the at least one densely sampled pharmacokinetic model as a target output for training the machine learning model 120.

Further, as described herein, the sparsely sampled pharmacokinetic model may include a plasma concentration of the molecule collected at a below-threshold frequency (e.g., a frequency that fails to meet the threshold frequency) and/or a below-threshold quantity (e.g., a quantity that fails to meet the threshold quantity) of pharmacokinetic measurements that is insufficient for describing the activity of the molecule after delivery to the patient. The below-threshold frequency may include pharmacokinetic measurements collected at every 12 hours to 24 hours, 24 hours to 48 hours, 24 hours to 1 week, 1 week to 2 weeks, 2 weeks to 4 weeks, and/or the like. The below-threshold quantity may include pharmacokinetic measurements collected at 1-2 time points, less than 3 time points, less than 4 time points and/or the like. Additionally and/or alternatively, the at least one sparsely sampled pharmacokinetic model may include a plurality of pharmacokinetic measurements, such as maximums (e.g., peaks) and minimums (e.g., troughs), following at least one dose of the molecule delivered to the patient that are not unique (e.g., sufficiently distinct from one another such that the values can be considered as part of the pharmacokinetic measurements collected at the above-threshold frequency and/or the above-threshold quantity). Thus, the at least one sparsely sampled pharmacokinetic parameter may be missing pharmacokinetic measurements and/or have an insufficient quantity of pharmacokinetic measurements (e.g., unique pharmacokinetic measurements) to accurately describe the overall activity of the molecule after delivery to the patient.

In some implementations, the at least one sparsely sampled pharmacokinetic model is synthetically generated, such as by the machine learning controller 110. For example, the at least one sparsely sampled pharmacokinetic model may be generated based on at least one densely sampled pharmacokinetic model. As an example, the at least one sparsely sampled pharmacokinetic model may be generated by removing at least one pharmacokinetic measurement from the at least one densely sampled pharmacokinetic model. The at least one pharmacokinetic measurement may be removed so that the at least one pharmacokinetic model has a below-threshold frequency and/or a below-threshold quantity of pharmacokinetic measurements.

Conventional machine learning models and non-compartmental analysis techniques may inaccurately determine one or more pharmacokinetic parameters based on sparsely sampled pharmacokinetic models. Consistent with implementations of the current subject matter, the architecture 200 provides for accurate prediction of one or more pharmacokinetic parameters based on sparsely sampled pharmacokinetic models. Thus, the architecture 200 allows for accurate pharmacokinetic parameter prediction even when the underlying pharmacokinetic measurements may be insufficient to describe the activity of the molecule after delivery of the molecule to the patient. As described herein, the machine learning controller 110 may train the machine learning model 120 based on the at least one sparsely sampled pharmacokinetic model and/or may apply the trained machine learning model 120 to determine one or more pharmacokinetic parameters based on the at least one sparsely sampled pharmacokinetic model.

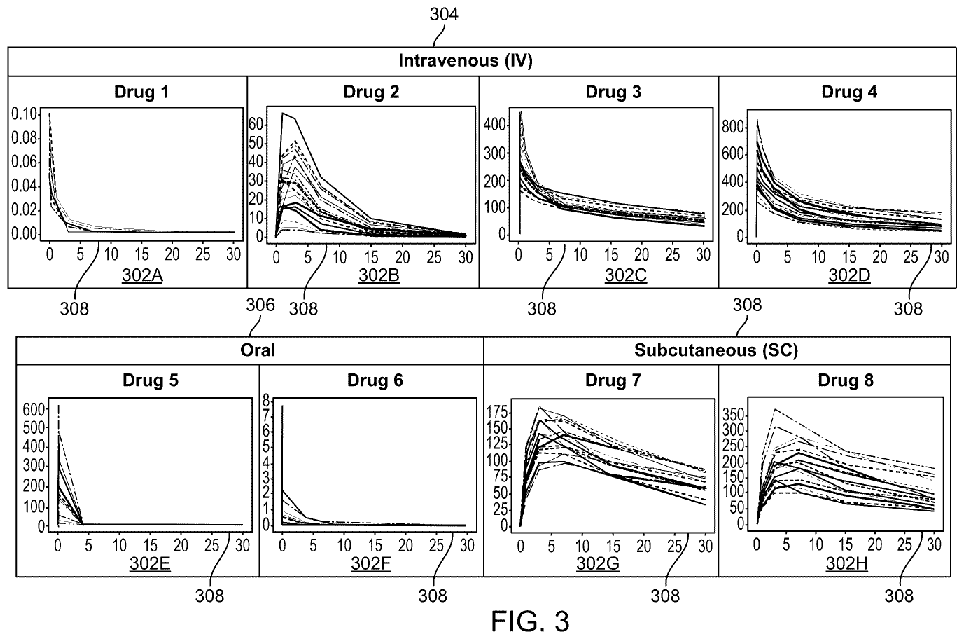

Each of the one or more pharmacokinetic models 242 may correspond to a particular patient. The one or more pharmacokinetic models 242 may additionally and/or alternatively correspond to a delivery method for delivering the molecule to the patient. For example, as shown in FIG. 3, the one or more pharmacokinetic models may correspond to a particular delivery method, including intravenous delivery 304, oral delivery 306, and subcutaneous delivery 308. Again referring to FIG. 3, the input 202 may include one or more pharmacokinetic profiles 302 including one or more pharmacokinetic models 242 corresponding to a particular molecule. For example, the input 202 may include a first pharmacokinetic profile 302A corresponding to a first molecule, a second pharmacokinetic profile 302B corresponding to a second molecule, a third pharmacokinetic profile 302C corresponding to a third molecule, a fourth pharmacokinetic profile 302D corresponding to a fourth molecule, a fifth pharmacokinetic profile 302E corresponding to a fifth molecule, a sixth pharmacokinetic profile 302F corresponding to a fifth molecule, a sixth pharmacokinetic profile 302F corresponding to a sixth molecule, a seventh pharmacokinetic profile 302G corresponding to a seventh molecule, an eighth pharmacokinetic profile 302H corresponding to an eighth molecule, and/or the like.

Additionally and/or alternatively, the input 202 includes one or more ground truth values for one or more pharmacokinetic parameters 255 (see FIG. 2B) associated with the one or more pharmacokinetic models. The one or more ground truth values may be determined by at least applying non-compartmental analysis to the one or more pharmacokinetic models, such as the at least one densely sampled pharmacokinetic model. The ground truth values may include at least one target pharmacokinetic parameter (e.g., a first target pharmacokinetic parameter 256, a second target pharmacokinetic parameter 258, a third target pharmacokinetic parameters 260, and/or the like) on which the machine learning controller 110 trains the machine learning model 120. The ground truth values may be used by the machine learning controller 110 as the prediction target output of the machine learning model 120 for training the machine learning model 120. The machine learning controller 110 may train the machine learning model 120 based on the ground truth target values to minimize an error between the determined value of a pharmacokinetic parameter and corresponding ground truth target value of the ground truth target values.

Referring to FIG. 2A, the preprocessing engine 150 may preprocess the one or more pharmacokinetic measurements 250 and/or the one or more target pharmacokinetic parameters (e.g., ground truth values). FIG. 2B schematically depicts a process 240 for preprocessing the one or more pharmacokinetic measurements 250 and/or the one or more target pharmacokinetic parameters 255, in accordance with some example embodiments. For example, the preprocessing engine 150 may normalize the values of the one or more pharmacokinetic measurements 250 and/or the one or more target pharmacokinetic parameters 255.

Generally, the values of the one or more pharmacokinetic measurements 250 and/or the one or more target pharmacokinetic parameters 255 have varying physical units of measurement that may result in inconsistent predictions made by machine learning models. For example, the one or more pharmacokinetic measurements 250 and/or the one or more target pharmacokinetic parameters 255 may have physical units of measurement, such as mg/L, g/L, g, mg, seconds, hours, days, and/or the like. Such variance in units can result in inconsistent predictions and/or increase computing requirements for making such predictions. Normalizing the values of the one or more pharmacokinetic measurements 250 and/or the one or more target pharmacokinetic parameters 255 helps to ensure unit-invariance of the one or more pharmacokinetic parameters determined by the machine learning model 120. For example, the values of the one or more pharmacokinetic measurements 250 and/or the one or more target pharmacokinetic parameters 255 may result in the input 202, including the one or more pharmacokinetic measurements 250 and/or the one or more target pharmacokinetic parameters 255, being unitless. Thus, normalizing the values of the one or more pharmacokinetic measurements 250 and/or the one or more target pharmacokinetic parameters 255 improves the accuracy and consistency of the predicted pharmacokinetic parameters generated by the machine learning model 120 and/or reduces computing resource requirements.

The preprocessing engine 150 may retrieve the one or more pharmacokinetic measurements 250 and/or the one or more target pharmacokinetic parameters 255 from the database 135 to normalize the one or more pharmacokinetic measurements 250 and/or the one or more target pharmacokinetic parameters 255. The one or more pharmacokinetic measurements 250 and/or the one or more target pharmacokinetic parameters 255 may be normalized in a patient-specific manner. For example, the preprocessing engine 150 may generate and apply a scaling factor 270 (see FIG. 2B), specific to a particular patient, to the values of the one or more pharmacokinetic measurements 250 and/or the one or more target pharmacokinetic parameters 255.

The one or more pharmacokinetic measurements 250 may be represented in the form of tuple Xᵢ={timeᵢⱼ, PKᵢⱼ, doseᵢⱼ}, with 1≤j≤mᵢ, for patient i, where j is a number of the value of the pharmacokinetic measurement (e.g., 1 corresponding to a first pharmacokinetic measurement, 2 corresponding to a second pharmacokinetic measurement, and so on) in the corresponding pharmacokinetic model 242, mᵢ is the quantity of pharmacokinetic measurements, timeᵢⱼ is a time point at which a pharmacokinetic measurement was collected, PKᵢⱼ is a plasma concentration collected at the time point timeᵢⱼ, and doseᵢⱼ is a dosage of the molecule associated with the plasma concentration PKᵢⱼ collected at the time point timeᵢⱼ.

The preprocessing engine 150 may normalize the one or more pharmacokinetic measurements 250 by at least generating a patient-specific scaling factor 270 and/or applying the patient-specific scaling factor 270 to the corresponding value of the pharmacokinetic measurement 250. For example, the preprocessing engine 150 may normalize a first value having a first unit of measurement and corresponding to a time point timeᵢⱼ (e.g., a first pharmacokinetic measurement 251 in this example) by at least applying a patient-specific time scaling factor (e.g., a first scaling factor 272 in this example) to the value of the time point timeᵢⱼ.

The preprocessing engine 150 may generate the patient-specific time scaling factor (e.g., the first scaling factor 272) by at least determining a maximum value of the time points of a plurality of values of the time points across the corresponding pharmacokinetic model (e.g., a first pharmacokinetic model 241). For example, the patient-specific time scaling factor is the total (e.g., maximum) time value during which pharmacokinetic measurements were collected to define the corresponding pharmacokinetic model. Here, the patient-specific time scaling factor scale_{time,i} may be determined as scale_{time,i}=maxⱼ (timeᵢⱼ).

The patient-specific time scaling factor scale_{time,i} may be applied (e.g., by the preprocessing engine 150) to the value of the time point to normalize the time point by at least dividing the value by the patient-specific time scaling factor scale_{time,i}. In other words, the value of the time point may be normalized by dividing the value of the time point by the patient-specific time scaling factor scale_{time,I} (e.g., the maximum value of the time point). This ensures that the normalized value of the time point for each of the plurality of time point values is unitless and at least zero and less than or equal to 1.

The preprocessing engine 150 may normalize a second value having a second unit of measurement and corresponding to a plasma concentration PKᵢⱼ (e.g., a second pharmacokinetic measurement 252 in this example) collected at the corresponding point timeᵢⱼ by at least applying a patient-specific plasma concentration scaling factor (e.g., a second scaling factor 274 in this example) to the value of the plasma concentration PKᵢⱼ.

The preprocessing engine 150 may generate the patient-specific plasma concentration scaling factor (e.g., the second scaling factor 274) by at least determining a maximum value of the plasma concentration of a plurality of values of the plasma concentrations across the corresponding pharmacokinetic model (e.g., the first pharmacokinetic model). For example, the patient-specific plasma concentration scaling factor is the total (e.g., maximum) plasma concentration during which pharmacokinetic measurements were collected to define the corresponding pharmacokinetic model. Here, the patient-specific plasma concentration scaling factor scale_{PK,i} may be determined as scale_{PK,i}=maxⱼ(PKᵢⱼ).

The patient-specific plasma concentration scaling factor scale_{PK,i} may be applied (e.g., by the preprocessing engine 150) to the value of the plasma concentration to normalize the value of the plasma concentration by at least dividing the value by the patient-specific plasma concentration scaling factor scale_{PK,i}. In other words, the value of the plasma concentration may be normalized by dividing the value of the plasma concentration by the patient-specific plasma concentration scaling factor scale_{PK,i} (e.g., the maximum value of the plasma concentration). This ensures that the normalized value of the plasma concentration for each of the plurality of plasma concentration values is unitless and at least zero and less than or equal to 1.

Moreover, the preprocessing engine 150 may normalize a third value having a third unit of measurement and corresponding to a dose doseᵢⱼ (e.g., a third pharmacokinetic measurement 254 in this example) collected at the corresponding point timeᵢⱼ by at least applying a patient-specific dose scaling factor (e.g., a third scaling factor 276 in this example) to the value of the dose doseᵢⱼ.

The preprocessing engine 150 may generate the patient-specific dose scaling factor (e.g., the third scaling factor 276) by at least determining a maximum value of the dose of a plurality of values of the dose across the corresponding pharmacokinetic model (e.g., the first pharmacokinetic model). For example, the patient-specific dose scaling factor is the total (e.g., maximum) dose during which doses were collected to define the corresponding pharmacokinetic model. Here, the patient-specific dose scaling factor scale_{dose,i} may be determined as scale_{dose,i}=maxⱼ(doseᵢⱼ).

The patient-specific dose scaling factor scale_{dose,i} may be applied (e.g., by the preprocessing engine 150) to the value of the dose to normalize the value of the dose by at least dividing the value by the patient-specific dose scaling factor scale_{dose,i}. In other words, the value of the dose may be normalized by dividing the value of the dose by the patient-specific dose scaling factor scale_{dose,i} (e.g., the maximum value of the dose). This ensures that the normalized value of the dose for each of the plurality of dose values is unitless and at least zero and less than or equal to 1.

Accordingly, the normalized values 232 of the one or more pharmacokinetic measurements 250 may be represented in the form of normalized tuple

$${\tilde{X}}_{ι} = \{ \frac{{time}_{ij}}{{scale}_{time , i}} , \frac{{PK}_{ij}}{{scale}_{PK , i}} , \frac{{dose}_{ij}}{{scale}_{dose , i}} \} .$$

Here, all components of the normalized tuple \acute{X}ₜ are unitless and are greater than or equal to zero and less than or equal to 1. The normalized tuple \tilde{X}ₜ may be stored in the database 135.

As noted, the preprocessing engine 150 may normalize the values of the one or more target output pharmacokinetic parameters 255 for use by the machine learning controller 110 in training the machine learning model 120. The preprocessing engine 150 may normalize the values of the one or more target pharmacokinetic parameters 255, such as a maximum plasma concentration of a molecule, a half-life of the molecule, and an area under curve of a plasma concentration curve associated with a pharmacokinetic model 242 (e.g., the first pharmacokinetic model 241 in this example). The preprocessing engine 150 normalizes the values of the one or more target parameters 255 such that the target half-life of the molecule, the target area under curve, and the target maximum plasma concentration are unitless.

The one or more target pharmacokinetic parameters 255 may be represented in the form of tuple

$${Y}_{i} = \{ {t}_{\frac{1}{2} , i} , {AUC}_{i} , C {max}_{i} \}$$

for patient i, where t_{1/2,i} (e.g., a first target pharmacokinetic parameter 256 in this example) is a target half-life of a molecule for a patient, AUCᵢ (e.g., a third target pharmacokinetic parameter 260 in this example) is an area under curve of a plasma concentration curve associated with a pharmacokinetic model of the patient, and Cmaxᵢ (e.g., a second target pharmacokinetic parameter 258 in this example) is a maximum plasma concentration of the molecule for the patient. In this example, the target half-life of the molecule has a physical unit of measurement of [time], the target area under curve has a physical unit of measurement of [concentration]×[time], and the target maximum plasma concentration has a physical unit of measurement [concentration]. Since the physical units of the values of the one or more target pharmacokinetic parameters 255, such as the target half-life of the molecule, the target area under curve, and the target maximum plasma concentration, are the same as the physical units of the time point and the plasma concentration pharmacokinetic measurements and/or are a function of the physical units of the time point and the plasma concentration pharmacokinetic measurements, the preprocessing engine 150 applies the patient-specific time scaling factor and/or the patient-specific plasma concentration scaling factor to the values of the one or more target pharmacokinetic parameters 255 having the corresponding physical unit of measurement.

For example, both the time point pharmacokinetic measurement and the target half-life of the molecule have the same physical unit of measurement, [time]. Thus, the preprocessing engine 150 may apply the patient-specific time scaling factor scale_{time,i} to the value of the target half-life of the molecule by, for example, dividing the value by the patient-specific time scaling factor scale_{time,i}. This ensures that the normalized value of the target half-life of the molecule is at least zero and less than or equal to 1. Similarly, both the plasma concentration pharmacokinetic measurement and the target maximum plasma concentration of the molecule have the same physical unit of measurement, [concentration]. Thus, the preprocessing engine 150 may apply the patient-specific plasma concentration scaling factor scale_{PK,i} to the value of the target maximum plasma concentration of the molecule by, for example, dividing the value by the patient-specific plasma concentration scaling factor scale_{PK,i}. This ensures that the normalized value of the target maximum plasma concentration of the molecule is at least zero and less than or equal to 1.

Moreover, the target area under curve has a physical unit of measurement of [concentration]×[time], which is a function of the physical unit of measurement, [concentration] of the plasma concentration pharmacokinetic measurement and the physical unit of measurement, [time] of the time point pharmacokinetic measurement. Thus, the preprocessing engine 150 may apply the patient-specific time scaling factor scale_{time,i} and the patient-specific plasma concentration scaling factor scale_{PK,i} to the value of the target area under curve by, for example, multiplying the patient-specific time scaling factor scale_{time,i} and the patient-specific plasma concentration scaling factor scale_{PK,i} and dividing the value of the target area under curve by the multiplied patient-specific time scaling factor scale_{time,i} and patient-specific plasma concentration scaling factor scale_{PK,i}. This ensures that the normalized value of the target area under curve of the molecule is at least zero and less than or equal to 1.

Accordingly, the normalized values 232 of the one or more target pharmacokinetic parameters 255 may be represented in the form of normalized target tuple

$${\tilde{Y}}_{ι} = \{ \frac{{t}_{\frac{1}{2} , i}}{{scale}_{time , i}} , \frac{A U {C}_{i}}{{scale}_{PK , i} \times {scale}_{t ime , i}} , \frac{C {max}_{i}}{{scale}_{PK , i}} \} .$$

Here, all components of the normalized target tuple [symbol] are unitless and are greater than or equal to zero and less than or equal to 1. The normalized target tuple \tilde{Y}ₗ may be stored in the database 135.

FIG. 2C schematically depicts a visual representation of the patient-specific normalization process 200C, consistent with implementations of the current subject matter. As discussed herein elsewhere, in order to achieve numerical invariance in the Deep-NCA model, irrespective of the physical units employed in the input data, a patient-specific normalization methodology may be developed to standardize both the input and output data. As discussed, the Deep-NCA model may be designed to handle a comprehensive range of pharmacokinetic (PK) data, as opposed to neural-PK/PD models, which are constructed from clinical trial data specific to particular treatments. Therefore, the implementation of unit equivariance (e.g., unit-invariance, unitless) may be particularly beneficial in enhancing the generalizability of deep learning models by reducing the impact of variable scaling on the learning task, thereby facilitating the network's ability to discern patterns across diverse datasets. As discussed herein elsewhere, to generate unitless set of input data and target variables, the PK data of each patient is scaled (e.g., divided) by the maximum value of each variable. As shown in FIG. 2C, the left upper panel illustrates the original PK data for patient i. The pre-processing engine 150 may process the original PK data and generate patient-specific normalized data associated with patient I, which is visualized in the upper right plot of FIG. 2C. In some embodiments, as shown by the bottom left plot of FIG. 2C, three key PK parameters of interest are illustrated, namely

$${Y}_{i} = \{ {t}_{\frac{1}{2} , i} , {AUC}_{i} , C {max}_{i} \} .$$

In some embodiments, as shown by the bottom right plot of FIG. 2C, this

$${Y}_{i} = \{ {t}_{\frac{1}{2} , i} , {AUC}_{i} , C {max}_{i} \}$$

may be scaled to be unites, and therefore ensures that the normalized value of the target tuple of the molecule is at least zero and less than or equal to 1.

Referring back to FIG. 2A, the architecture 200 includes at least one convolutional layer 210 and at least one recurrent neural network unit 220. The at least one recurrent neural network unit 220 follows the at least one convolutional layer 210 in the architecture. The at least one convolutional layer 210 receives the input 202, such as from the database 135. The at least one convolutional layer 210 may optionally receive the normalized values 232 of the input 202, such as the normalized pharmacokinetic measurements underlying the one or more pharmacokinetic models 242, and/or the one or more normalized target pharmacokinetic parameters, from the database 135 and/or the preprocessing engine 150.

The at least one convolutional layer 210 includes uses at least one convolutional kernel to extract one or more features from the input 202 (e.g., one or more pharmacokinetic models associated with a molecule) and/or the input 202 including the normalized values 232, and/or to generate a feature map based on the extracted one or more features. For example, the at least one convolutional layer 210 may extract, from the one or more pharmacokinetic models 242, one or more features at at least one time point (e.g., a first time point, a second time point, and/or the like) after the molecule has been delivered to the patient. Based on the extracted one or more features, the at least one convolutional layer 210 may predict a value (e.g., values 215) of a pharmacokinetic parameter, such as an area under curve, at the corresponding time points. For example, the at least one convolutional layer 210 may predict at least one value 215, including a first value of the pharmacokinetic parameter, such as the area under curve, at a first time point, a second value of the pharmacokinetic parameter at a second time point, a third value of the pharmacokinetic parameter at the third time point, and/or the like. In this manner, the at least one convolutional layer 210 may employ the at least one convolutional kernel to capture a snapshot in time indicating the area under curve at various time points in the snapshot of time.

The at least one convolutional layer 210 may output the values 215 (e.g., the first value, the second value, the third value, and/or the like). The at least one recurrent neural network unit 220 may receive the outputted values 215. Based on the received values 215 (e.g., (e.g., the first value, the second value, the third value, and/or the like), the at least one recurrent neural network unit 220 may determine a value of another pharmacokinetic parameter, such as a maximum plasma concentration of the molecule and/or a half-life of the molecule.

The at least one convolutional layer 210 may include at least two convolutional layers, such as a first convolutional layer 212 and a second convolutional layer 214. The at least one convolutional layer 210 may further include a third convolutional layer, a fourth convolutional layer, and so on. As noted, the at least one convolutional layer 210, such as the first convolutional layer 212 and/or the second convolutional layer 214 may include at least one convolutional kernel to extract the one or more features from the one or more pharmacokinetic models. The first convolutional layer 212 and/or the second convolutional layer 214 may include a plurality of channels (e.g., 20 channels, 10 to 20 channels, or more) for processing the input 202.

The first convolutional layer 212 may extract a first plurality of features 211 from the one or more pharmacokinetic models and the second convolutional layer 214 may extract a second plurality of features 213 from the one or more pharmacokinetic models. For example, the second convolutional layer 214 may receive the extracted first plurality of features 211 from the first convolutional layer 212 and extract the second plurality of features 213 based at least one the first plurality of extracted features. The architecture of the at least one convolutional layer 210 allows for accurate predictions of the pharmacokinetic parameters, such as the area under curve, and for a plurality (e.g., two or more) area under curve pharmacokinetic parameters to be predicted by the at least one convolutional layer 210.

The at least one recurrent neural network unit 220 may receive and concatenate the extracted features and/or feature maps from the at least one convolutional layer. For example, the at least one recurrent neural network unit 220 may propagate the predicted values of the first pharmacokinetic parameter and/or the extracted features at the various time points from the at least one convolutional layer 210 over time across the time series concentration data of the one or more pharmacokinetic models of the input 202. Thus, the at least one recurrent neural network unit 220 may compare the extracted features and/or predicted values of the first pharmacokinetic parameter (e.g., the area under curve) across various time points. Based on the comparison, the at least one recurrent neural network unit 220 may determine at least one other pharmacokinetic parameter (e.g., a second pharmacokinetic parameter and/or a third pharmacokinetic parameter), such as the maximum plasma concentration and/or the half-life of the molecule.

The at least one recurrent neural network unit 220 may include a first recurrent neural network unit 222, a second recurrent neural network unit 224, and so on. The first recurrent neural network unit 222 and the second recurrent neural network unit 224 may be a first hidden layer and/or a second hidden layer, respectively. The first recurrent neural network unit 222 and/or the second recurrent neural network unit 224 may include at least one memory cell, allowing the at least one recurrent neural network unit 220 to propagate the extracted features from the at least one convolutional layer 210 over time. The first recurrent neural network unit 222 and the second recurrent neural network unit 224 may be coupled to a dense layer 226, which may, alone, or together with the first recurrent neural network unit 222 and/or the second recurrent neural network unit 224 predict the value of the second pharmacokinetic parameter based on the values of the first pharmacokinetic parameter from the at least one convolutional layer 210.

The architecture 200, including the at least one convolutional layer 210 and/or the at least one recurrent neural network unit 220 allows for the input 202 to include an arbitrary length sequence with varying quantities of dimensions (e.g., sampled pharmacokinetic measurements at various quantities of time points). Thus, the architecture 200 processes inputs 202 having varying quantities of samples, and may not be limited to a particular quantity of samples.

Referring again to FIG. 2A, the machine learning controller 110 may apply the trained machine learning model 120 to determine a value of a plurality of pharmacokinetic parameters (e.g., a first pharmacokinetic parameter, a second pharmacokinetic parameter, and/or a third pharmacokinetic parameter), such as an area under curve, a half-life, and/or a maximum plasma concentration, based on at least one pharmacokinetic model, such as at least one sparsely sampled pharmacokinetic model. Accordingly, the machine learning model 120 may accurately predict values of one or more pharmacokinetic parameters in circumstances in which the underlying input data is incomplete and/or includes a sparse quantity of pharmacokinetic measurements.

As discussed herein elsewhere, the present disclosure provides a deep learning model, referred to herein as Deep-NCA, for performing Non-Compartmental Analysis (NCA) of pharmacokinetic (PK) data. Traditional NCA algorithms, while extensively employed, often exhibit reduced accuracies when dealing with sparse PK samples. In response to this, Deep-NCA, a deep learning (DL) model, has been developed to enhance the prediction of non-compartmental PK parameters. In some embodiments, this methodology leverages synthetic PK data for model training and implements a patient-specific normalization technique for data preprocessing. The model has demonstrated satisfactory performance across multiple simulated drugs under various dosing conditions, indicating effective generalization. In comparison to traditional NCA, Deep-NCA has shown superior performance for sparse PK data. This disclosure advances the application of deep learning to PK studies and introduces a method for handling sparse PK data. As such, Deep-NCA may substantially enhance the efficiency of drug development by providing more accurate NCA estimates while requiring fewer PK samples.

Non-compartmental analysis (NCA) is a widely utilized technique for pharmacokinetic (PK) analysis that involves determining the PK parameters of a drug based on its concentration-time curve measurements. As discussed herein elsewhere, despite its widespread application, traditional NCA algorithms suffer from limitations that may affect the accuracy of parameter estimation and its domain of applicability. One notable constraint lies in the dependence on relatively dense PK measurements, which may not be practical or achievable in specific patient populations such as pediatrics, rare diseases, and animal studies. Conversely, the use of sparse PK samples can pose a challenge to traditional NCA approaches, which may lead to inaccurate estimation of drug exposure and clearance parameters. Moreover, NCA relies on pre-determined analytical methods which may not work well in some settings. For example, the trapezoidal rule, which is frequently employed to calculate area under the curve (AUC), can result in under- or over-estimation of the AUC on sparse PK measurements. Finally, traditional NCA is a semi-manual rather than a fully automated process which relies on the competency of the PK analyst which may introduce subjectivity and additional variability.

Deep learning (DL) models have emerged as a tool for pharmacometric analysis, with the potential for offering advantages over NCA analysis. DL models have shown an ability to handle sparse time sequence data, which is relevant in clinical trials where there may be limited PK samples available. Furthermore, DL models have demonstrated the ability to glean insights from historical datasets to tackle challenges. By analyzing patterns in these datasets, DL models can provide a comprehensive recognition of PK properties and improve the accuracy of parameter predictions. Furthermore, DL models automate the evaluation process, hence eliminating the need for human intervention, thus reducing subjectivity. Moreover, unlike traditional NCA approaches which are fixed analytical methodologies, DL models offer the potential for continuous optimization through ongoing algorithm evolution and training data enrichment, resulting in enhanced model performance and improved accuracy of PK parameter predictions over time.

This subject matters disclosed herein presents Deep-NCA, a study that employs DL to infer NCA parameters, with the aim of addressing the limitations inherent in conventional NCA. Deep-NCA is designed to leverage the strengths of DL models, including their ability to handle sparse data, learn from large compiled datasets, reduce subjectivity through automation, and continuously optimize for better performance through iterative algorithm development. Positioned at the intersection of PK and DL, Deep-NCA aims to provide a solution to the complex challenges of modern PK analysis. The design, implementation, and potential benefits of Deep-NCA are discussed in further details herein elsewhere.

In some embodiments, in order to generate training data for the Deep-NCA model, a variety of pharmacokinetic (PK) profiles may be simulated. Dosing scenarios considered include the administration of large molecules via intravenous (IV) and subcutaneous (SC) routes, and the oral administration of small molecules. For each dosing scenario, a substantial number of distinct PK profiles (e.g., 100,000, 300,000, 500,000, 1 million, 2 million, 3 million, 4 million, 5 million, 10 million, 100 million, etc.) may be simulated using a standard two-compartment model. The model parameters may be uniformly distributed to reflect the typical range encountered in a two-compartment PK model for each dosing scenario. To ensure a diverse parameter distribution, the boundaries for representative drugs may be computed to determine the individual PK parameter range. For example, to guarantee a diverse parameter distribution, the boundaries for a number of representative drugs (in this study, N=8) are computed to determine the individual PK parameter range (P_{low}, P_{high}) for the synthetic data, based on their typical values obtained from available population PK (popPK) models (see Equations (2) and (3)). Employing these boundaries, the PK parameter ranges of training data for both large and small molecules are determined as shown in Table 1.

$${P}_{i}^{95 \% C I} = θ \times exp ( \pm 1 \text{.96} \times sqrt ( {ω}^{2} ) ) \qquad ( 1 )$$

$${P}_{low} = α \times min {\{ ( {P}_{i}^{95 \% C I} ) \}}_{i = 1}^{N} , α = 0.5 \qquad ( 2 )$$

$${P}_{high} = β \times min {\{ ( {P}_{i}^{95 \% C I} ) \}}_{i = 1}^{N} , β = 0.5 \qquad ( 3 )$$

where Pᵢ^{95% Cl} is 95% confidence interval of individual PK parameter for drug i derived from popPK model, θ is the typical value, ω² is the inter-individual variability on the log scale for the parameter of interest, P_{low} is the lower boundary of PK parameter for synthetic data, P_{high} is upper boundary of PK parameter for synthetic data, α=0.5 and β=1.5 are extension factors to further extend the boundaries to cover extreme PK profile. In order to ensure a more realistic set of parameter combinations, an empirically chosen correlation of p=0.2 was imposed between central volume of distribution (V1), clearance (CL), inter-compartmental clearance (Q), and peripheral volume of distribution (V2) using the correlate package.

TABLE 1
PK parameter ranges used to generate the training
data for large and small molecules.

| | Large molecules | | | Small molecules | |
|---|---|---|---|---|---|
| | Lower | Upper | | Lower | Upper |
| | bound- | bound- | | bound- | bound- |
| | ary | ary | | ary | ary |
| CL(L/day) | 0.05 | 3 | CL(L/day) | 0.67 | 2700 |
| V1 (L) | 1 | 15 | V1/F (L) | 0.49 | 4900 |
| V2 (L) | 0.5 | 20 | V2/F (L) | 3.7 | 2100 |
| Q (L/day) | 0.5 | 5 | Q/F (L/day) | 48 | 8000 |
| Ka (1/day) | 0.05 | 0.8 | Ka (1/day) | 2 | 110 |
| ALAGI (day) | N/A | N/A | ALAG1 (day) | 0 | 0.125 |
| F1 (for SC | 0.2 | 0.9 | | | |
| only) | | | | | |

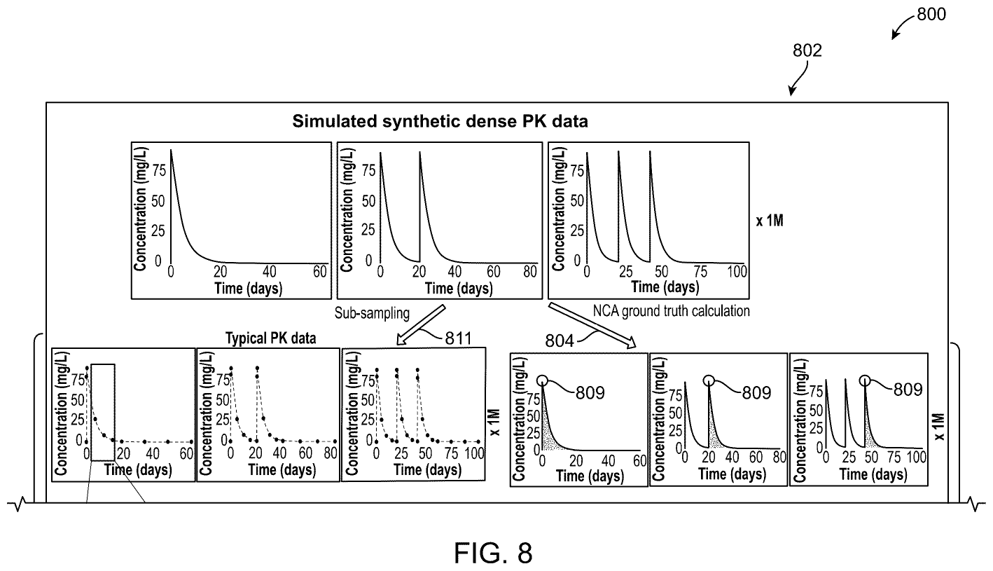

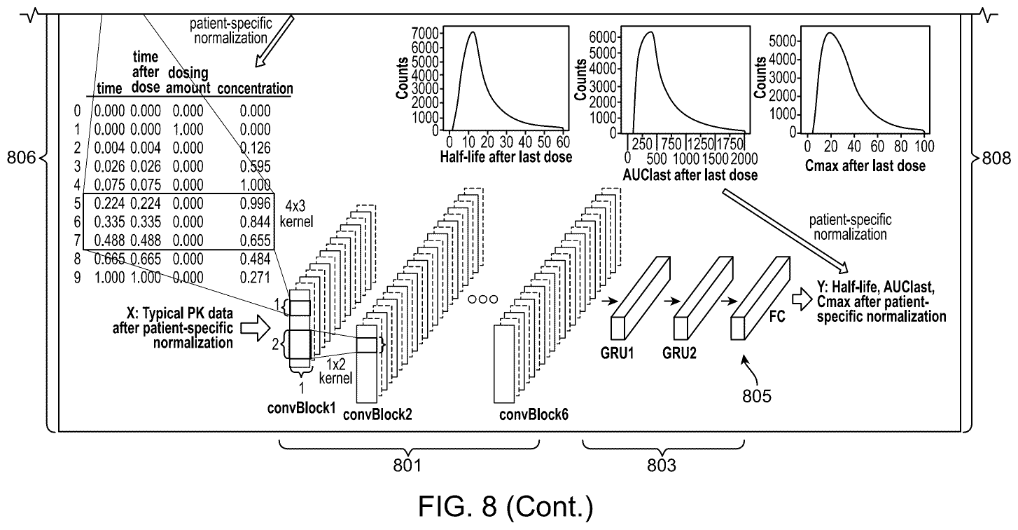

FIG. 8 depicts an architecture 800 illustrating a pharmacokinetic prediction system, for example, the architecture 800 from synthetic PK data generation to model training, consistent with implementations of the current subject matter. As shown in FIG. 8, dense PK profiles may be generated with an hourly sampling rate (FIG. 8, 802). In the case of large molecule simulation data, in some embodiments, a 21-day dosing interval (Q3 W) may be employed, with a maximum of three doses, and the duration of PK measurement after the final dose extends up to 63 days. For small molecule data, a daily (QD) dosing may be employed, with a maximum of five doses (this allows the model to handle multiple doses without requiring the PK to reach steady-state) and PK measurement extending up to seven days after the last dose. Ground truth NCA for three key PK parameters after the last dose: namely, half-life, area under the curve after last dose (AUClast), and peak plasma concentration (Cmax), is established by employing PKNCA as calculated from the dense PK profiles (FIG. 8, right arrow 804). In some embodiments, these NCA parameter values computed from the dense PK profiles serve as prediction targets for the Deep-NCA model.

As shown in FIG. 8, the workflow from synthetic PK data generation to model training is illustrated using a training set consisting of a set of 1 million (1 M) PK data for large molecules. The actions involved may comprise: synthetic dense PK data generation, the calculation of ground truth prediction targets, typical PK (tPK) data subsampling from the dense PK data, patient-specific normalization of both the PK data and the corresponding targets (e.g., target valuables), and/or subsequent Deep-NCA model training. As shown in FIG. 8, the left branch 806 of the figure depicts the sub-sampling process and patient-specific normalization of tPK data, with the sub-sampling time points (TPs) denoted by the darker dots. The right branch 808 represents the NCA ground truth calculation and corresponding patient-specific normalization of NCA targets. The Cmax are represented by the circles 809, while the AUClast (Area Under the Curve from the last dose to the final TP measured) are shown as shaded areas following the last dose. Key components of the Deep-NCA model include ConvBlock (convolution block) 801, GRU (Gated Recurrent Units) 803, and FC (Fully Connected Layers) 805. To emulate the typical pharmacokinetic (tPK) data obtained in clinical trials, the process may begin by simulating dense PK profiles. From these profiles, discretized time bins are established based on the Time After Dose (TAD). For each of these time bins, one PK sample per bin for each dose is randomly selected (as shown in the left arrow 811 of the figure). These bins are designed differently for large and small molecules to ensure a thorough characterization of the true PK curve. As such, each set of bins is constructed to capture the absorption, maximum concentration, distribution phase, trough (pre-dose) sampling, and terminal phase characterization for the final dose. Following this, pairs of tPK and ground truth NCA data are used as training data for the Deep-NCA model.

As discussed herein elsewhere, a Convolutional Recurrent Neural Network (CRNN) may be utilized to estimate PK parameters from tPK profiles that had undergone patient-specific normalization. As shown in FIG. 8, a proposed CRNN architecture may be constructed with six convolutional layers 801 and two gated recurrent unit (GRU) layers 803. The first convolutional layer 801 consisted of 100 channels, while each of the subsequent five convolutional layers 801 were comprised of 200 channels. Distinct kernel sizes may be used for the initial layer (4, 3) and the subsequent layers (1,2), coupled with the non-linear activation function, Leaky Rectified Linear Unit (Leaky ReLU). Kaiming initialization may be used for the layer weights, and biases may be set to zero. Following the convolutional segment of the model, two GRU layers 803 with 200 hidden units each may be incorporated. The GRU output may be transformed to three output classes through a fully connected (FC) linear layer 805. For example, the three output classes as shown in FIG. 8 may be half-life, AUClast, and Cmax after patient-specific normalization. In some embodiments, the output classes may be passed through a quantile transformer layer (not shown in FIG. 8) to map the output distribution into a Gaussian distribution to mitigate the effect of outliers on model generalization. For the training process, in some embodiments, a stochastic optimizer can be employed, for example the Adam optimizer or other suitable type of stochastic optimizer. The Adam optimizer is a first-order gradient-based optimization of stochastic objective functions, based on adaptive estimates of lower-order moments. In some embodiments, the Adam optimizer may be employed, using a learning rate of 0.0001 with a batch size of 1000.

In order to account for the distinct pharmacokinetic characteristics of small and large molecules (e.g., different distributions of clearance and volume of distribution), two Deep-NCA models were separately trained on PK parameter distributions that may be tailored to these respective drug classes. A data augmentation technique may be subsequently employed to enhance the generalization of the DL model by training on both the original dense tPK data as well as the sparsified versions. The sparsified tPK data may be generated by randomly omitting up to 4 TPs per dosing interval, as discussed elsewhere herein. To preserve tPK data points that would realistically be present in a PK study, the first TP, the maximum drug concentration TP, and the final TP for each dosing interval may be retained. The predictive performance of the Deep-NCA model may be evaluated against the traditional NCA model using three statistical measures: R2 (coefficient of determination), normalized root mean square deviation (nRMSE), and mean absolute percentage error (MAPE).

### Model Testing on Simulated Data

The predictive performance and generalization of the Deep-NCA may be evaluated by simulating a test dataset. This dataset may comprise six anonymized in-house drugs, each associated with a distinct pharmacokinetic profile and a population of 30,000 patients. In a manner analogous to the synthesis of training data, pairs of typical pharmacokinetic data and corresponding ground truth are generated for all six actual drugs. In some embodiments, this generation process may employ the corresponding population pharmacokinetic model. To assess the proficiency of the Deep-NCA model in managing sparse pharmacokinetic profiles, a random omission of up to four time points per dosing interval may be performed on the typical pharmacokinetic data. This process may result in the creation of sparse pharmacokinetic profiles. For benchmarking purposes, a traditional non-compartmental analysis may be performed on the sparsified pharmacokinetic data using the PKNCA method. The performance of this traditional analysis serves as a comparison point for evaluating the performance of the Deep-NCA in predicting pharmacokinetic parameters. Table 2 shows the description of the test datasets for the Deep-NCA model evaluation.

TABLE 2

| Drug ID | 1 | 2 | 3 | 4 | 5 | 6 |
|---|---|---|---|---|---|---|
| Molecule | mAb | ADC | mAb | mAb | Small | Small |
| format | | | | | molecule | molecule |
| Number of | 2 | 2 | 2 | 2 with | 2 | 3 |
| compartment | | | | Michaelis- | | |
| | | | | Menten | | |
| | | | | clearance | | |
| Route of | iv, sc | iv | iv, sc | iv | oral | oral |
| administration | | | | | | |

As shown in Table 3, a comparison of the predictive performance, assessed using R2, between traditional Non-Compartmental Analysis (NCA) and Deep-NCA for six drugs is presented. The traditional NCA approach displays excellent performance in NCA estimation, which can be attributed to the relatively dense sampling of typical pharmacokinetic (tPK) data. The predictive performance of the Deep-NCA model is also assessed using R2. The Deep-NCA model exhibits adequate performance across all six drugs and three prediction targets, as evidenced by the relatively high accuracy and consistency in predictions for both small and large molecules. This demonstrates the robust predictive abilities of the Deep-NCA model, indicating effective generalization across a diverse range of drug characteristics. Notably, it is underscored that the drug ID6 acts as an out-of-distribution validation, given that it is simulated by a 3-compartment model, in contrast to the 2-compartment model utilized for simulating the data that trained the Deep-NCA. This discrepancy might explain the imperfect prediction performance of half-life observed for drug ID6.

TABLE 3

| | Performance of | | | Performance of | | |
|---|---|---|---|---|---|---|
| | traditional NCA (R²) | | | deep-NCA (R²) | | |
| | half-life | AUClast | Cmax | half-life | AUClast | Cmax |
| Drug ID1 | 1.00 | 1.00 | 1.00 | 0.99 | 0.99 | 1.00 |
| Drug ID2 | 1.00 | 1.00 | 1.00 | 0.92 | 0.93 | 1.00 |
| Drug ID3 | 1.00 | 1.00 | 1.00 | 0.98 | 0.99 | 1.00 |
| Drug ID4 | 0.99 | 1.00 | 1.00 | 0.94 | 0.98 | 1.00 |
| Drug ID5 | 1.00 | 1.00 | 1.00 | 0.98 | 1.00 | 0.99 |
| Drug ID6 | 0.99 | 1.00 | 1.00 | 0.86 | 0.99 | 1.00 |

The performance of both Deep-NCA models is further evaluated on sparse pharmacokinetic (PK) data with increasing levels of sparsity, for example, up to a maximum of 4 time points (TPs) per dosing interval. In the traditional Non-Compartmental Analysis (NCA) of typical PK (tPK) data, the predictive performance, as measured by R², declines with the increasing sparsification, as shown in Table 4. As the sparsity levels increase, the calculation of half-life and AUCₗₐₛₜ via the R package PKNCA becomes increasingly challenging. This difficulty is manifested in the declining performance with increasing sparsity, and the increasing percentage of tPK data that PKNCA fails to estimate (i.e., the returned outputs are NaNs), especially for drug IDs ID1, ID3, ID5, and ID6. The root cause of this difficulty may be the challenge of defining the terminal clearance in an automated manner. In contrast, the performance of Deep-NCA exhibits minor deterioration with increasing sparsity levels, and the model maintains its predictive capabilities. These results illustrate that the Deep-NCA model outperforms the traditional NCA method in the context of sparse PK data by consistently delivering precise estimates.

TABLE 4

| | | Performance of traditional NCA (R²) | | | | Performance of deep-NCA (R²) | | | |
|---|---|---|---|---|---|---|---|---|---|
| | | Number of remaining TPs after last dose | | | | | | | |
| | | 9 | 8 | 7 | 6 | 9 | 8 | 7 | 6 |
| Drug ID1 | half-life | 1.00 | 0.97 | 0.90 | 0.35 | 0.99 | 0.99 | 0.99 | 0.99 |
| | | | (0.2%) | (14.5%) | (47.1%) | | | | |
| | AUClast | 0.99 | 0.98 | 0.94 | 0.86 | 0.99 | 0.99 | 0.99 | 0.93 |
| | Cmax | 1.00 | 0.99 | 0.978 | 0.95 | 1.00 | 1.00 | 1.00 | 1.00 |
| Drug ID2 | half-life | 1.00 | 0.99 | 0.96 | 0.89 | 0.93 | 0.92 | 0.88 | 0.86 |
| | AUClast | 0.99 | 0.96 | 0.90 | 0.77 | 0.96 | 0.97 | 0.95 | 0.82 |
| | Cmax | 1.00 | 1.00 | 1.00 | 1.00 | 1.00 | 1.00 | 1.00 | 1.00 |
| Drug ID3 | half-life | 1.00 | 1.00 | 0.98 | 0.46 | 0.99 | 0.99 | 0.99 | 0.99 |
| | | | (0.02%) | (12.8%) | (69.7%) | | | | |
| | AUClast | 0.99 | 0.97 | 0.93 | 0.85 | 0.99 | 0.99 | 0.99 | 0.91 |
| | Cmax | 1.00 | 0.99 | 0.99 | 0.97 | 1.00 | 1.00 | 1.00 | 1.00 |
| Drug ID4 | half-life | 0.99 | 0.97 | 0.93 | 0.73 | 0.94 | 0.94 | 0.93 | 0.94 |
| | AUClast | 0.99 | 0.97 | 0.92 | 0.80 | 0.98 | 0.98 | 0.97 | 0.91 |
| | Cmax | 1.00 | 1.00 | 1.00 | 1.00 | 1.00 | 1.00 | 1.00 | 1.00 |
| Drug ID5 | half-life | 1.00 | 1.00 | 0.89 | 0.58 | 0.97 | 0.97 | 0.98 | 0.97 |
| | | (0.05%) | (0.4%) | (2.6%) | (14.7%) | | | | |
| | AUClast | 0.99 | 0.97 | 0.93 | 0.82 | 1.00 | 1.00 | 1.00 | 1.00 |
| | Cmax | 0.98 | 0.97 | 0.95 | 0.93 | 0.95 | 1.00 | 1.00 | 1.00 |
| Drug ID6 | half-life | 0.67 | −0.38 | −3.52 | −8.63 | 0.84 | 0.78 | 0.68 | 0.82 |
| | | | (0.04%) | (1.0%) | (8.9%) | | | | |
| | AUClast | 0.99 | 0.96 | 0.89 | 0.73 | 0.99 | 0.99 | 0.99 | 0.98 |
| | Cmax | 0.99 | 0.99 | 0.98 | 0.97 | 1.00 | 0.99 | 1.00 | 1.00 |

The present subject matter introduces Deep-NCA, an innovative approach designed to address the challenges of traditional Non-Compartmental Analysis (NCA). This methodology offers multiple advantages. Firstly, the training for Deep-NCA utilizes extensive data simulated by population pharmacokinetic (popPK) models, thereby endowing it with a comprehensive understanding of PK profiles. Consequently, Deep-NCA has the potential to achieve the accuracy of NCA obtained from popPK modeling, considered as the “gold-standard”, without the extensive effort and time typically associated with model development, selection, and validation. Furthermore, while traditional NCA requires human intervention, especially when determining the terminal phase, deep learning (DL) models offer an efficient, automated alternative. The integration of DL with NCA retains its simplicity of use while enabling the handling of sparse PK data to generate accurate results. In the development of DL applications, the choice of model architectures is made with consideration for the computation that is to be performed on the data. The Convolutional Recurrent Neural Network (CRNN) architecture is selected due to its distinctive ability to manage time sequence data of variable lengths, a pivotal attribute when dealing with PK data. This architecture combines the desirable attributes of convolutional networks in performing local computations, such as the calculation of the area of a trapezoid, with that of Recurrent Neural Networks (RNNs) in performing global computation by leveraging a sequence of local computations of arbitrary lengths. To enhance the model's ability to comprehend the dosing interval naturally inherent in PK data, the feature of Time After Dose (TAD) is incorporated into the PK data. This is intended to demonstrate that the Deep-NCA model can utilize not just the PK information subsequent to the last dose but also effectively leverage prior PK data by borrowing information from analogous TAD intervals. The impact of input and target preprocessing on DL models' stability, performance, and convergence is considerable, particularly when handling large training datasets. In this study, the combined impact of two preprocessing techniques, patient-specific normalization and quantile transformer, is applied. A patient-specific normalization approach is first used to standardize input data, promoting model generalization and consistency across different physical units. This method is utilized for both input and target variables, scaling the PK data and the PK parameters to facilitate pattern discernment across diverse PK profiles and render the scaled targets independent of the choice of units. Subsequently, the quantile transformation method is employed on the patient-specific normalized target variable, which proves effective in enhancing DL models' performance through mitigation of sensitivity to outliers and noise. Notably, the models with the quantile transformer show better predictive performance compared to models without the quantile transformer. The effect of these methods, when combined in a preprocessing pipeline, leads to improved stability, accelerated convergence, and enhanced generalization performance in the DL model, demonstrating the advantages of integrating complementary techniques for preprocessing data in complex modeling tasks. Future iterations of Deep-NCA may utilize more sophisticated training data, such as the PK data with residual variability, as well as the integration of advanced DL architectures and algorithms for enhanced performance and adaptability.

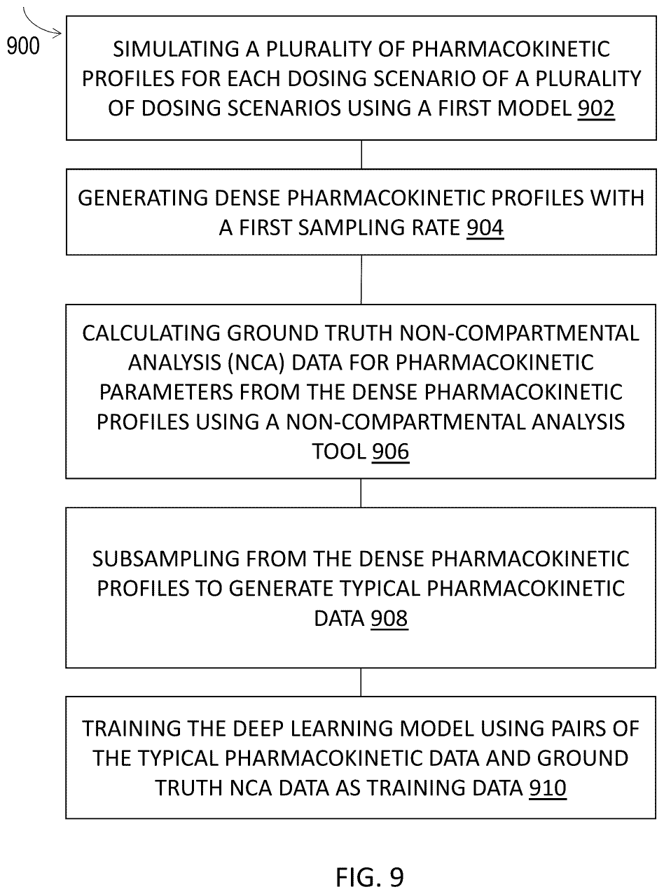

FIG. 9 depicts a flowchart illustrating an example of a process for training a deep learning model, consistent with implementations of the current subject matter. As shown in FIG. 9, the machine learning controller (e.g., the machine learning controller 110) may train a deep learning model (e.g., the machine learning model 120) using synthetic PK data. As discussed herein elsewhere, the process 900 may begin with operation 902, wherein the system may simulate a plurality of pharmacokinetic profiles for each dosing scenario of a plurality of dosing scenarios using a first model. In some embodiments, the dosing scenarios comprise a 21-day dosing interval for large molecules and a daily dosing for small molecules. In some embodiments, the dosing scenarios comprise intravenous, subcutaneous, and oral administration of a molecule. Next, the process 900 may proceed to operation 904, wherein the system may generate dense pharmacokinetic profiles with a first sampling rate. In some embodiments, the first sample rate may be determined based on the pharmacokinetic properties of the drug being simulated. For instance, drugs with a fast absorption and elimination rate may require a higher sampling rate to accurately capture their pharmacokinetic profiles. Conversely, drugs with slower absorption and elimination rates may require a lower sampling rate. The dense pharmacokinetic profiles generated at this stage provide a comprehensive representation of the drug's behavior in the body over time, capturing the full dynamics of drug absorption, distribution, metabolism, and excretion. This dense data may serve as the foundation for the subsequent steps in the process, including the calculation of ground truth non-compartmental analysis (NCA) data and the creation of sparse pharmacokinetic profiles for model training.

Next, the process 900 may proceed to operation 906, wherein the system may calculating ground truth non-compartmental analysis (NCA) data for pharmacokinetic parameters from the dense pharmacokinetic profiles using a non-compartmental analysis tool. In some embodiments, the non-compartmental analysis tool calculates parameters such as the half-life of the drug, the area under the concentration-time curve (AUC), and the peak plasma concentration (Cmax). These parameters may provide insights into the pharmacokinetic behavior of the drug, serving as the ground truth against which the predictions of the deep learning model are compared. The non-compartmental analysis tool may use standard pharmacokinetic equations and methods to calculate these parameters, ensuring that the ground truth data is accurate and reliable. In some cases, the tool may also account for factors such as the dosing regimen, the route of administration, and the patient population, providing a comprehensive and detailed analysis of the pharmacokinetic data.

Next, the process 900 may proceed to operation 908, wherein the system may subsample from the dense pharmacokinetic profiles to generate typical pharmacokinetic data. In some embodiments, the subsampling operation 908 may comprise the establishment of discretized time bins based on the Time After Dose (TAD). For each of these time bins, one pharmacokinetic sample is randomly selected for each dose. This approach ensures a balanced representation of the pharmacokinetic profile across the different time points post-dose, thereby preserving the overall shape and characteristics of the pharmacokinetic profiles. The subsampling process creates a set of sparse pharmacokinetic profiles that reflect the realities of data collection in a clinical setting, where it may not be feasible or practical to collect dense pharmacokinetic data. In some cases, the system may be configured to control the level of sparsity in the typical pharmacokinetic data by adjusting the number of time points omitted during the subsampling process. This allows the system to generate typical pharmacokinetic data that matches the specific requirements and constraints of different clinical scenarios.

Next, the process 900 may proceed to operation 910, wherein the system may train the deep learning model using pairs of the typical pharmacokinetic data and ground truth NCA data. For example, the system may feed the deep learning model with the typical pharmacokinetic data as input and the corresponding ground truth NCA data as the target output. The deep learning model, which may be a Convolutional Recurrent Neural Network (CRNN), learns to map the input data to the target output, thereby learning to predict non-compartmental pharmacokinetic parameters based on the typical pharmacokinetic data. In some embodiments, the training process and/or operation 910 may comprise the use of a suitable optimization algorithm, such as the Adam optimizer, to iteratively adjust the model's parameters to minimize the difference between the model's predictions and the ground truth NCA data. The system may also employ techniques such as batch normalization and dropout to improve the model's generalization performance and prevent overfitting. In some embodiments, the training process may continue until the model's performance on a validation set of data reaches a satisfactory level, at which point the model is ready to be used for predicting non-compartmental pharmacokinetic parameters from new pharmacokinetic data.

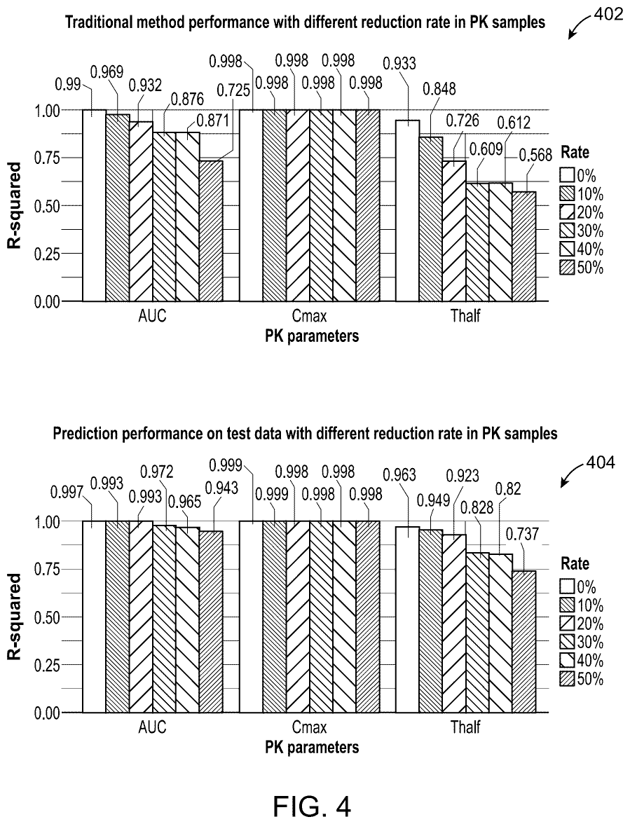

FIG. 4 depicts a performance comparison between the machine learning model 120 and a conventional method for determining one or more pharmacokinetic parameters, consistent with implementations of the current subject matter. As shown in FIG. 4, a bar graph 402 depicts a performance of a conventional non-compartmental analysis method for determining one or more pharmacokinetic parameters and a bar graph 404 depicts a performance of the machine learning model 120, consistent with implementations of the current subject matter, for determining one or more pharmacokinetic parameters. As shown in the comparison of the bar graph 402 and the bar graph 404, the traditional non-compartmental analysis method (shown in the bar graph 402) experiences a greater loss in accuracy in determining pharmacokinetic parameters when sparse data is available compared to the machine learning model 120 (shown in the bar graph 404).

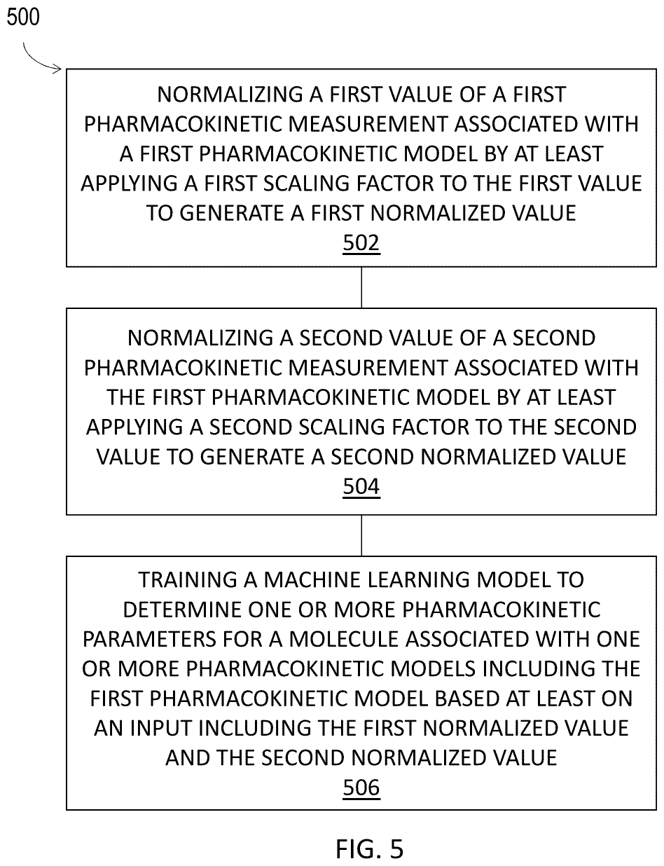

FIG. 5 depicts a flowchart illustrating an example of a process 500 for preprocessing input data (e.g., the input data 202) for training a machine learning model (e.g., the machine learning model 120), consistent with implementations of the current subject matter. The process 500 is described with reference to FIGS. 2A and 2B. The process 500 may be implemented by the preprocessing engine 150. The process 500 may be performed to normalize at least a portion of the input data 202, such as one or more pharmacokinetic measurements 250 (e.g., a time point, a plasma concentration measured at the time point, a dosage measured at the time point, and/or the like) of time series concentration data defining one or more pharmacokinetic models 242 (e.g., a first pharmacokinetic model 241, a second pharmacokinetic model 243, a third pharmacokinetic model 245, and/or the like) and/or one or more target pharmacokinetic parameters 255 (e.g., a target maximum plasma concentration of a molecule, a half-life of the molecule, and an area under curve of a plasma concentration curve for the molecule). The one or more pharmacokinetic measurements may be generated by at least applying non-compartmental analysis to the one or more pharmacokinetic models. Referring to FIG. 2B, the one or more pharmacokinetic measurements 250 may include a first pharmacokinetic measurement 251, a second pharmacokinetic measurement 252, a third pharmacokinetic measurement 254, and/or the like. Again referring to FIG. 2B, the one or more target pharmacokinetic parameters 255 may include a first target pharmacokinetic parameter 256, a second target pharmacokinetic parameter 258, a third target pharmacokinetic parameter 260, and/or the like.

For example, the one or more pharmacokinetic measurements 250 may be normalized, such that the one or more normalized values 232 of the pharmacokinetic measurements 250 is unitless. This ensures the unit-invariance of the output (e.g., the output 230) of the machine learning model 120, thereby allowing for more efficient processing of the input, reducing computational resource requirements, and improving the accuracy of the output. Consistent with implementations of the current subject matter, the process 500 refers to the example architecture 200 shown in FIG. 2A and the preprocessing process 240 shown in FIG. 2B.

Referring to FIG. 5, at 502, the preprocessing engine 150 normalizes a first value of a first pharmacokinetic measurement 251 associated with a first pharmacokinetic model 241 (e.g., at least a portion of the input 202) by at least applying a first scaling factor 272 to the first value to generate a first normalized value 280. The first value is in a first unit of measurement (e.g., mg/L, g/L, g, mg, seconds, hours, days, etc.), and the first normalized value 280 is unitless. Thus, the preprocessing engine 150 normalizes the first value of the first pharmacokinetic measurement 251 so that the first normalized value 280 is unitless.

As an example, the first pharmacokinetic measurement 251 is one of a time point, a plasma concentration measured at the time point, a dosage measured at the time point, and/or the like. In some implementations, the first scaling factor 272 is a first maximum value of a plurality of first values of the first pharmacokinetic measurement 251 across the first pharmacokinetic model 241. The plurality of first values of the first pharmacokinetic measurement 251 may correspond to various time points in the first pharmacokinetic model 241. The first scaling factor 272 may be applied to the first pharmacokinetic measurement 251 to normalize the first pharmacokinetic measurement 251 by at least dividing the first value by the first scaling factor 272. In other words, the first value of the first pharmacokinetic measurement 251 may be normalized by dividing the first value by the first maximum value. This ensures that the first normalized value 280 for each of the plurality of first values is at least zero and less than or equal to 1.

At 504, the preprocessing engine 150 normalizes a second value of a second pharmacokinetic measurement 252 associated with the first pharmacokinetic model 241 (e.g., at least a portion of the input 202) by at least applying a second scaling factor 274 to the second value to generate a second normalized value 282. The second value is in a second unit of measurement (e.g., mg/L, g/L, g, mg, seconds, hours, days, etc.), and the second normalized value 282 is unitless. Thus, the preprocessing engine 150 normalizes the second value of the second pharmacokinetic measurement 252 so that the second normalized value 282 is unitless.

As an example, the second pharmacokinetic measurement 252 is another one of a time point, a plasma concentration measured at the time point, a dosage measured at the time point, and/or the like. For example, the first pharmacokinetic measurement 251 may be one of the time point, the plasma concentration, and the dosage, and the second pharmacokinetic measurement 252 may be one of the remaining ones of the time point, the plasma concentration, and the dosage.

In some implementations, the second scaling factor 274 is a second maximum value of a plurality of second values of the second pharmacokinetic measurement 252 across the first pharmacokinetic model 241. The plurality of second values of the second pharmacokinetic measurement 252 may correspond to various time points in the first pharmacokinetic model 241. The second scaling factor 274 may be applied to the second pharmacokinetic measurement 252 to normalize the second pharmacokinetic measurement 252 by at least dividing the second value by the second scaling factor 274. In other words, the second value of the second pharmacokinetic measurement 252 may be normalized by dividing the second value by the second maximum value. This ensures that the normalized second value for each of the plurality of second values is at least zero and less than or equal to 1.

In some implementations, the preprocessing engine 150 normalizes a third value of a third pharmacokinetic measurement 254 associated with the first pharmacokinetic model 241 (e.g., at least a portion of the input 202) by at least applying a third scaling factor 276 to the third value to generate a third normalized value 284. The third value is in a third unit of measurement (e.g., mg/L, g/L, g, mg, seconds, hours, days, etc.), and the third normalized value 284 is unitless. Thus, the preprocessing engine 150 normalizes the third value of the third pharmacokinetic measurement 254 so that the third normalized value 284 is unitless.

As an example, the third pharmacokinetic measurement 254 is another one of a time point, a plasma concentration measured at the time point, a dosage measured at the time point, and/or the like. For example, the first pharmacokinetic measurement 251 may be one of the time point, the plasma concentration, and the dosage, the second pharmacokinetic measurement 252 may be one of the remaining ones of the time point, the plasma concentration, and the dosage, and the third pharmacokinetic measurement 254 may be one of the remaining ones of the time point, the plasma concentration, and the dosage, such as one that is not the first pharmacokinetic measurement 251 and/or the second pharmacokinetic measurement 252.

In some implementations, the third scaling factor 276 is a third maximum value of a plurality of third values of the third pharmacokinetic measurement 254 across the first pharmacokinetic model 241. The plurality of third values of the third pharmacokinetic measurement 254 may correspond to various time points in the first pharmacokinetic model 241. The third scaling factor 276 may be applied to the third pharmacokinetic measurement 254 to normalize the third pharmacokinetic measurement 254 by at least dividing the third value by the third scaling factor 276. In other words, the third value of the third pharmacokinetic measurement 254 may be normalized by dividing the third value by the third maximum value. This ensures that the third normalized value 284 for each of the plurality of third values is at least zero and less than or equal to 1.

At 506, the machine learning controller 110 may train the machine learning model 120 to determine one or more pharmacokinetic parameters for a molecule associated with one or more pharmacokinetic models 242 including the first pharmacokinetic model 241 based at least on an input 202 including the first normalized value 280 and the second normalized value 282. The molecule is at least one of a micro molecule having a molecular weight of less than 1000 Daltons and a macro molecule having a molecular weight of greater than or equal to 1000 Daltons. The one or more pharmacokinetic parameters may include an area under curve of a plasma concentration curve associated with the one or more pharmacokinetic models, a maximum plasma concentration of the molecule, a half-life of the molecule, and/or the like. The machine learning controller 110 may train the machine learning model 120 using the processes 600, 650, consistent with implementations of the current subject matter.

In some implementations, the machine learning controller 110 further trains the machine learning model 120 based on a target output, such as a target value of a target pharmacokinetic parameter 255. The target value of the target pharmacokinetic parameter 255 may be normalized by the preprocessing engine 150 prior to the machine learning controller 110 training the machine learning model 120 based on the target value of the target pharmacokinetic parameter. Normalization of the target value of the target pharmacokinetic parameters 255 is described with reference to FIGS. 2A and 2B.

For example, the preprocessing engine 150 may normalize a first target value of a first target pharmacokinetic parameter 256 associated with the first pharmacokinetic model 241 by at least applying the first scaling factor 272 to the first target value of the first target pharmacokinetic parameter 256. In this example, the first target pharmacokinetic parameter 256 is one of a maximum plasma concentration of the molecule and a half-life of the molecule. Further, in this example, the first pharmacokinetic measurement 251 is a corresponding one of the time point and the plasma concentration. Thus, the first unit of measurement may be the same as the first target unit of measurement associated with the first target value, and the normalized first target value 286 is unitless. The preprocessing engine 150 normalizes the first target value of the first target pharmacokinetic parameter so that the first target value is unitless. The first scaling factor 272 may be applied to the first target value to normalize the first target value of the first pharmacokinetic parameter 256 by at least dividing the first target value by the first scaling factor. This ensures that the normalized first target value 286 is at least zero and less than or equal to 1.

In some implementations, the preprocessing engine 150 may normalize a second target value of a second target pharmacokinetic parameter 258 associated with the first pharmacokinetic model 241 by at least applying the second scaling factor 274 to the second target value of the second target pharmacokinetic parameter 258. In this example, the second target pharmacokinetic parameter 258 is one of a maximum plasma concentration of the molecule and a half-life of the molecule. Further, in this example, the second pharmacokinetic measurement 252 is a corresponding one of the time point and the plasma concentration. Thus, the second unit of measurement may be the same as the second target unit of measurement associated with the second target value, and the normalized second target value 288 is unitless. The preprocessing engine 150 normalizes the second target value of the second target pharmacokinetic parameter 258 so that the normalized second target value 288 is unitless. The second scaling factor 274 may be applied to the second target value to normalize the second target value of the second pharmacokinetic parameter 256 by at least dividing the second target value by the second scaling factor 274. This ensures that the normalized second target value 288 is at least zero and less than or equal to 1.

In some implementations, the preprocessing engine 150 may normalize a third target value of a third target pharmacokinetic parameter 260 associated with the first pharmacokinetic model 241 by at least applying the third scaling factor 276 to the third target value of the third target pharmacokinetic parameter 260. In this example, the third target pharmacokinetic parameter 260 is an area under curve of a plasma concentration curve associated with the first pharmacokinetic model 241. The preprocessing engine 150 normalizes the third target value of the third target pharmacokinetic parameter 260 so that the normalized third target value 289 is unitless. The third scaling factor 276 in this example may be generated by at least multiplying the first scaling factor 272 (e.g., a scaling factor associated with a time measurement and/or a scaling factor associated with a plasma concentration measurement) and the second scaling factor 274 (e.g., the other one of the scaling factor associated with the time measurement and/or the scaling factor associated with the plasma concentration measurement). The third scaling factor 276 in this example may be applied to the third target value to normalize the third target value of the third target pharmacokinetic parameter 260 by at least dividing the third target value by the third scaling factor 276. This ensures that the normalized third target value 289 is at least zero and less than or equal to 1.

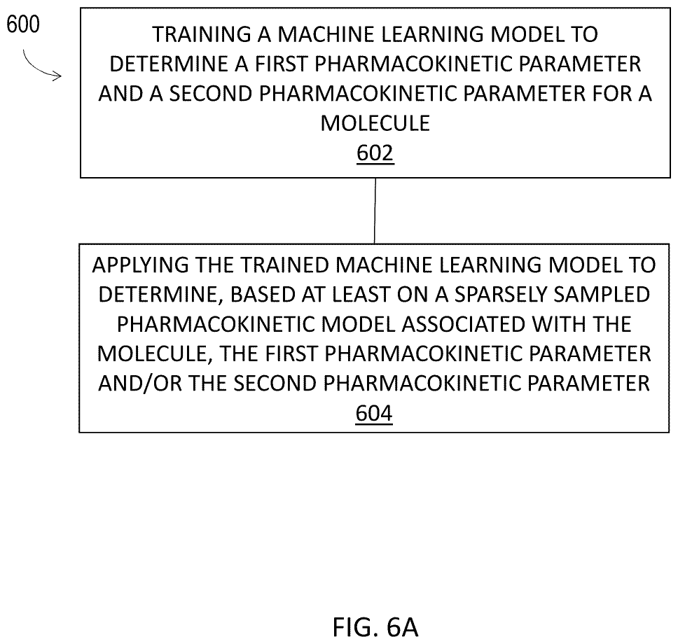

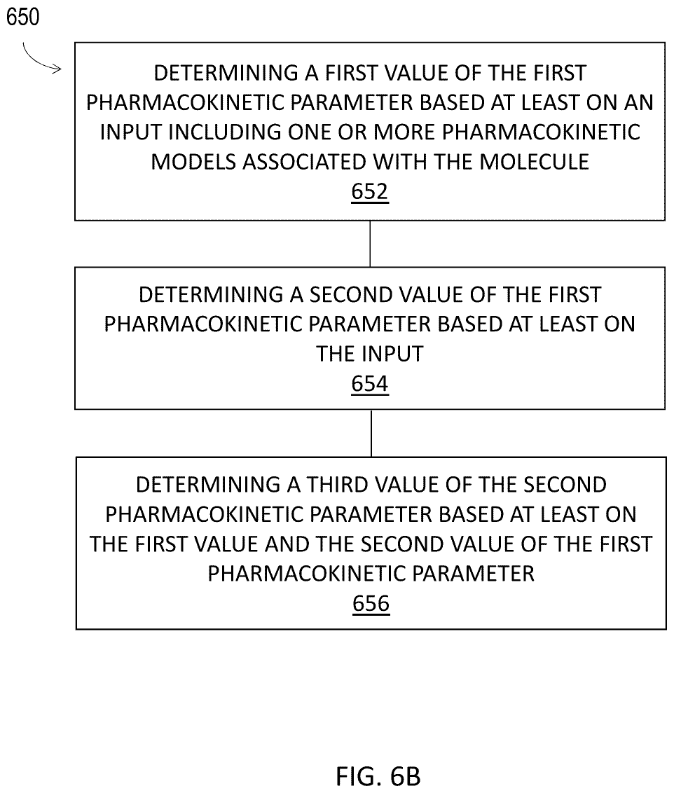

FIG. 6A and FIG. 6B depict flowcharts illustrating an example of processes 600, 650 for deep learning for non-compartmental analysis, consistent with implementations of the current subject matter. Referring to FIG. 6A and FIG. 6B, the processes 600, 650 may be performed by the machine learning controller 110 to determine one or more pharmacokinetic parameters for a pharmacokinetic model 242, such as a sparsely sampled pharmacokinetic model. The machine learning controller 110 may thus train the machine learning model 120 to determine one or more pharmacokinetic parameters with improved accuracy, efficiency, and a reduction in computational resource requirements. Consistent with implementations of the current subject matter, the processes 600, 650 refer to the example architecture 200 shown in FIG. 2A and the preprocessing process 240 shown in FIG. 2B.

Referring to FIG. 6A, at 602, the machine learning controller (e.g., the machine learning controller 110) may train a machine learning model (e.g., the machine learning model 120) to determine a first pharmacokinetic parameter and a second pharmacokinetic parameter for a molecule. The molecule is at least one of a micro molecule having a molecular weight of less than 1000 Daltons and a macro molecule having a molecular weight of greater than or equal to 1000 Daltons. The first pharmacokinetic parameter may include an area under curve of a plasma concentration curve associated with one or more pharmacokinetic models. The second pharmacokinetic parameter is at least one of a maximum plasma concentration of the molecule and/or a half-life of the molecule.

The machine learning controller 110 may train the machine learning model 120 based at least on an input, such as the input 202. The input 202 may include one or more pharmacokinetic measurements (e.g., the pharmacokinetic measurements 250), one or more pharmacokinetic models (e.g., the plurality of models 242), and/or one or more target pharmacokinetic parameters (e.g., the target pharmacokinetic parameters 255).

The one or more pharmacokinetic measurements may include a plurality of time points, a plasma concentration measured at each of the plurality of time points, and/or a dosage measured at each of the plurality of time points. Corresponding pharmacokinetic measurements (e.g., a time point, the plasma concentration measured at the time point, and a dosage measured at the time point) may be included in the input 202 as a tuple. Thus, the input 202 may include a plurality of tuples, each including a time point, a concentration, and a dosage. The plurality of tuples represent the time series concentration data of each pharmacokinetic model of the one or more pharmacokinetic models. In some implementations, the values of each of the pharmacokinetic measurements can be normalized such that the normalized values are unitless. The values of the pharmacokinetic measurements may be normalized by, for example, implementing the process 500, as described herein.

As noted, the input 202 may additionally and/or alternatively include one or more target pharmacokinetic parameters. As described herein, the one or more target pharmacokinetic parameters may be determined by at least applying non-compartmental analysis techniques to the one or more pharmacokinetic models including the one or more pharmacokinetic measurements. The one or more target pharmacokinetic parameters may include ground truth target values based on which the machine learning controller 110 trains the machine learning model 120. For example, the machine learning controller 110 may train the machine learning model 120 to minimize an error or loss between a value of the pharmacokinetic parameters (e.g., the first pharmacokinetic parameter, the second pharmacokinetic parameter, and/or the like) determined by the machine learning model 120 and the corresponding target pharmacokinetic parameter of the one or more target pharmacokinetic parameters. In some implementations, the values of each of the one or more target pharmacokinetic parameters can be normalized such that the normalized values are unitless. The values of the target pharmacokinetic parameters may be normalized by, for example, implementing the process 500, as described herein.

Referring to FIG. 6B, the machine learning controller 110 may train the machine learning model 120 using process 650. For example, the machine learning controller 110 may, based at least on the input 202 including the one or more pharmacokinetic models, the one or more pharmacokinetic measurements, and/or the one or more target pharmacokinetic parameters, train the machine learning model 120 to determine one or more pharmacokinetic parameters (e.g., a first pharmacokinetic parameter, a second pharmacokinetic parameter, a third pharmacokinetic parameter, and/or the like), such as an area under curve of a plasma concentration curve, a maximum plasma concentration, and a half-life for a molecule.

At 652, the machine learning controller 110 may determine a first value of the first pharmacokinetic parameter based at least on an input (e.g., the input 202) including one or more pharmacokinetic models 242 associated with the molecule. The first value may correspond to a first area under curve at a first time point. The first value may be determined by at least one convolutional layer (e.g., the at least one convolutional layer 210) of the machine learning model 120. The at least one convolutional layer 210 may include two convolutional layers, such as the first convolutional layer 212 and/or the second convolutional layer 214.

The input may be preprocessed (e.g., normalized) by the preprocessing engine 150, such as by using the process 500 and/or the process 240, consistent with implementations of the current subject matter. For example, the machine learning controller 110 may receive the normalized input, such as the normalized values 232 of the pharmacokinetic measurements and/or the normalized target values of the target pharmacokinetic parameters from the preprocessing engine 150.

The one or more pharmacokinetic models 242 of the input 202 includes time series data of a plasma concentration collected at a plurality of time points after the molecule is delivered to a patient. The time series concentration of data may include a variable quantity of the plurality of time points. Thus, the input is not limited to a certain quantity of the time series concentration data collected at a particular quantity of the plurality of time points. The time series concentration data includes a plurality of tuples, such as a first tuple, a second tuple, and/or the like. The first tuple includes a first time point of the plurality of time points, a first plasma concentration measured at the first time point, and/or a first dosage measured at the first time point. The second tuple includes a second time point of the plurality of time points, a second plasma concentration measured at the second time point, and a second dosage measured at the second time point, and so on. As noted, the time series concentration data, upon which the machine learning model 120 is trained, may not be limited to a certain quantity of tuples.

The one or more pharmacokinetic models 242 of the input 202 may include a densely sampled pharmacokinetic model and/or a sparsely sampled pharmacokinetic model. The densely sampled pharmacokinetic model may include a plasma concentration of the molecule collected at an above-threshold frequency (e.g., greater than two tuples, five tuples, ten tuples, and/or the like). The sparsely sampled pharmacokinetic model may include a plasma concentration of the molecule collected at a below-threshold frequency (e.g., less than two tuples, one to two tuples, and/or the like).

In some implementations, the input 202 further includes one or more ground truth values for the first pharmacokinetic parameter and/or the second pharmacokinetic parameter associated with the one or more pharmacokinetic models 242. The one or more ground truth values (e.g., the values of the one or more target pharmacokinetic parameters 255) may be determined by at least applying non-compartmental analysis to the one or more pharmacokinetic models 242. The one or more ground truth values may be preprocessed (e.g., normalized) by the preprocessing engine 150 using the process 500 and/or the process 240, consistent with implementations of the current subject matter.

At 654, the machine learning controller 110 may determine a second value of the first pharmacokinetic parameter based at least on the input 202. The second value may correspond to a second area under curve at a second time point. The second time point may be later than the first time point. The second value may be determined by the at least one convolutional layer 210 of the machine learning model 120. The at least one convolutional layer may output the first value and/or the second value of the first pharmacokinetic parameter. At least one recurrent neural network unit (e.g., the recurrent neural network unit 220) may receive the first value and the second value. The at least one recurrent neural network unit 220 may include two recurrent neural network units, such as a first recurrent neural network unit 222, and/or a second recurrent neural network unit 224.

At 656, the machine learning controller 110 may determine a third value of the second pharmacokinetic parameter based at least on the first value and the second value of the first pharmacokinetic parameter. The third value of the second pharmacokinetic parameter may be determined by the at least one recurrent neural network unit 220 of the machine learning model 120. The machine learning controller 110 may further train the machine learning model 120 by at least minimizing an error between the third value of the second pharmacokinetic parameter and a ground truth target value of the one or more ground truth target values corresponding to the second pharmacokinetic parameter.

In some implementations, the machine learning controller 110 may determine a fourth value of a third pharmacokinetic parameter based at least on the first value and the second value of the first pharmacokinetic parameter. The fourth value of the third pharmacokinetic parameter may be determined by the at least one recurrent neural network unit 220 of the machine learning model 120. The third pharmacokinetic parameter may be one of the maximum plasma concentration of the molecule and the half-life of the molecule. For example, the second pharmacokinetic parameter may include one of the maximum plasma concentration of the molecule and the half-life of the molecule, and the third pharmacokinetic parameter may include the remaining one of the maximum plasma concentration of the molecule and the half-life of the molecule.

Referring again to FIG. 6A, at 604, the machine learning controller 110 may apply the trained machine learning model 120 to determine the first pharmacokinetic parameter and/or the second pharmacokinetic parameter based at least on a sparsely sampled pharmacokinetic model associated with the molecule. The sparsely sampled pharmacokinetic model associated with the molecule may include a concentration curve across a plurality of doses of the molecule. The concentration curve associated with the sparsely sampled pharmacokinetic model may include a single trough and/or peak. The concentration curve associated with the sparsely sampled pharmacokinetic model may include a plurality of troughs and/or peaks having a value that is approximately the same and/or within a threshold range of values. Thus, the trained machine learning model 120 may efficiently and/or accurately determine the first pharmacokinetic parameter and/or the second pharmacokinetic parameter for pharmacokinetic models that have limited available data.

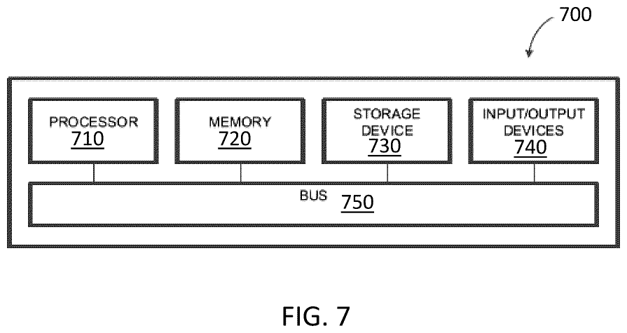

FIG. 7 depicts a block diagram illustrating a computing system 700 consistent with implementations of the current subject matter. Referring to FIGS. 1-7, the computing system 700 can be used to implement the machine learning controller 110, the preprocessing engine 150, the machine learning model 120, and/or any components therein.

As shown in FIG. 7, the computing system 700 can include a processor 710, a memory 720, a storage device 730, and input/output devices 740. The processor 710, the memory 720, the storage device 730, and the input/output devices 740 can be interconnected via a system bus 750. The computing system 700 may additionally or alternatively include a graphic processing unit (GPU), such as for image processing, and/or an associated memory for the GPU. The GPU and/or the associated memory for the GPU may be interconnected via the system bus 750 with the processor 710, the memory 720, the storage device 730, and the input/output devices 740. The memory associated with the GPU may store one or more images described herein, and the GPU may process one or more of the images described herein. The GPU may be coupled to and/or form a part of the processor 710. The processor 710 is capable of processing instructions for execution within the computing system 700. Such executed instructions can implement one or more components of, for example, the machine learning controller 110, the machine learning model 120, the preprocessing engine 150, and/or the like. In some implementations of the current subject matter, the processor 710 can be a single-threaded processor. Alternately, the processor 710 can be a multi-threaded processor. The processor 710 is capable of processing instructions stored in the memory 720 and/or on the storage device 730 to display graphical information for a user interface provided via the input/output device 740.

The memory 720 is a computer readable medium such as volatile or non-volatile that stores information within the computing system 700. The memory 720 can store data structures representing configuration object databases, for example. The storage device 730 is capable of providing persistent storage for the computing system 700. The storage device 730 can be a floppy disk device, a hard disk device, an optical disk device, or a tape device, or other suitable persistent storage means. The input/output device 740 provides input/output operations for the computing system 700. In some implementations of the current subject matter, the input/output device 740 includes a keyboard and/or pointing device. In various implementations, the input/output device 740 includes a display unit for displaying graphical user interfaces.

According to some implementations of the current subject matter, the input/output device 740 can provide input/output operations for a network device. For example, the input/output device 740 can include Ethernet ports or other networking ports to communicate with one or more wired and/or wireless networks (e.g., a local area network (LAN), a wide area network (WAN), the Internet).

In some implementations of the current subject matter, the computing system 700 can be used to execute various interactive computer software applications that can be used for organization, analysis and/or storage of data in various (e.g., tabular) format (e.g., Microsoft Excel®, and/or any other type of software). Alternatively, the computing system 700 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 740. The user interface can be generated and presented to a user by the computing system 700 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

What is claimed is:

1. A method, comprising:
training a machine learning model to determine a first pharmacokinetic parameter and a second pharmacokinetic parameter for a molecule by at least:
determining, based at least on an input including one or more pharmacokinetic models associated with the molecule, a first value of the first pharmacokinetic parameter,
determining, based at least on the input, a second value of the first pharmacokinetic parameter, and
determining, based at least on the first value and the second value of the first pharmacokinetic parameter, a third value of the second pharmacokinetic parameter; and
applying the trained machine learning model to determine, based at least on a sparsely sampled pharmacokinetic model associated with the molecule, the first pharmacokinetic parameter and/or the second pharmacokinetic parameter.

2. The method of claim 1, wherein the input further includes one or more ground truth target values for the first pharmacokinetic parameter and/or the second pharmacokinetic parameter associated with the one or more pharmacokinetic models, wherein the one or more ground truth values are determined by at least applying non-compartmental analysis to the one or more pharmacokinetic models, and wherein the machine learning model is further trained by at least minimizing an error between the third value of the second pharmacokinetic parameter and a ground truth target value of the one or more ground truth target values corresponding to the second pharmacokinetic parameter.

3. The method of claim 1, wherein the one or more pharmacokinetic models includes time series data of a plasma concentration of the molecule collected at a plurality of time points after the molecule is delivered to a patient.

4. The method of claim 3, wherein the time series concentration data includes: a first tuple including a first time point of the plurality of time points, a plasma concentration measured at the first time point, and a dosage measured at the first time point; and a second tuple including a second time point of the plurality of time points, a second plasma concentration measured at the second time point, and a second dosage measured at the second time point.

5. The method of claim 1, wherein the first value is determined at a first time point; and wherein the second value is determined at a second time point after the first time point.

6. The method of claim 5, wherein the first pharmacokinetic parameter is an area under curve (AUC) of a plasma concentration curve associated with the one or more pharmacokinetic models.

7. The method of claim 6, wherein the first value corresponds to a first area under curve at the first time point; and wherein the second value corresponds to a second area under curve at the second time point.

8. The method of claim 1, wherein the second pharmacokinetic parameter is at least one of a maximum plasma concentration of the molecule and a half-life of the molecule.

9. The method of claim 1, wherein the first value and the second value are determined by at least one convolutional layer of the machine learning model.

10. The method of claim 9, wherein the third value is determined by at least one recurrent neural network unit of the machine learning model.

11. The method of claim 10, wherein the machine learning model is further trained to determine the one or more pharmacokinetic parameters for the molecule by at least: outputting, by the at least one convolutional layer, the first value and the second value; and receiving, by the at least one recurrent neural network unit, the first value and the second value.

12. The method of claim 1, wherein the machine learning model is further trained to determine the one or more pharmacokinetic parameters for the molecule by at least determining, based on the first value and the second value, a fourth value of a third pharmacokinetic parameter of the one or more pharmacokinetic parameters.

13. The method of claim 1, wherein the molecule is a micro molecule having a molecular weight of less than 1000 Daltons.

14. The method of claim 1, wherein the molecule is a macro molecule having a molecular weight of greater than or equal to 1000 Daltons.

15. The method of claim 1, wherein the sparsely sampled pharmacokinetic model associated with the molecule includes a concentration curve across a plurality of doses of the molecule.

16. The method of claim 1, further comprising normalizing a unit of measurement associated with the one or more pharmacokinetic models.

17. The method of claim 1, wherein the one or more pharmacokinetic models includes a densely sampled pharmacokinetic model in which a plasma concentration of the molecule is collected at an above-threshold frequency.

18. The method of claim 1, wherein the one or more pharmacokinetic models includes a sparsely sampled pharmacokinetic model in which a plasma concentration of the molecule is collected at a below-threshold frequency.

19. A system comprising: one or more processors; and a non-transitory memory coupled to the processors comprising instructions executable by the processors, the processors operable when executing the instructions to:
train a machine learning model to determine a first pharmacokinetic parameter and a second pharmacokinetic parameter for a molecule by at least:
determining, based at least on an input including one or more pharmacokinetic models associated with the molecule, a first value of the first pharmacokinetic parameter,
determining, based at least on the input, a second value of the first pharmacokinetic parameter, and
determining, based at least on the first value and the second value of the first pharmacokinetic parameter, a third value of the second pharmacokinetic parameter; and
apply the trained machine learning model to determine, based at least on a sparsely sampled pharmacokinetic model associated with the molecule, the first pharmacokinetic parameter and/or the second pharmacokinetic parameter.

20. One or more computer-readable non-transitory storage media embodying software that is operable when executed to:
train a machine learning model to determine a first pharmacokinetic parameter and a second pharmacokinetic parameter for a molecule by at least:
determining, based at least on an input including one or more pharmacokinetic models associated with the molecule, a first value of the first pharmacokinetic parameter,
determining, based at least on the input, a second value of the first pharmacokinetic parameter, and
determining, based at least on the first value and the second value of the first pharmacokinetic parameter, a third value of the second pharmacokinetic parameter; and
apply the trained machine learning model to determine, based at least on a sparsely sampled pharmacokinetic model associated with the molecule, the first pharmacokinetic parameter and/or the second pharmacokinetic parameter.
